(19) United States
(12) Patent Application Publication
(10) Pub. No.: US 2026/0216220 A1
(43) Pub. Date: Jul. 30, 2026

(54) TREATMENT OF MYELOPEROXIDASE-POSITIVE ANCA-ASSOCIATED VASCULITIS WITH H2S RELEASING COMPOUNDS

(71) Applicant: DEBRECENI EGYETEM, Debrecen (HU)

(72) Inventors: Péter NAGY, Szentendre (HU); József BALLA, Debrecen (HU); Dorottya GARAI, Budapest (HU)

(21) Appl. No.: 19/142,342

(22) PCT Filed: Dec. 27, 2023
(86) PCT No.: PCT/HU2023/050098

§ 371 Date: Jun. 23, 2025

(30) Foreign Application Priority Data
Dec. 23, 2022 (HU) P2300003
Mar. 3, 2023 (HU) P2300079

(51) Int. Cl. A61K 31/675; A61P 9/14

(52) U.S. Cl. CPC A61K 31/675; A61P 9/14

(57) ABSTRACT

Treatment of Myeloperoxidase-positive ANCA-associated vasculitis with H2S releasing compounds The invention is related to hydrogen sulfide (H2S) releaser compounds and compositions for use in the treatment of myeloperoxidase (MPO) induced neutrophil mediated tissue damage, in particular positive antineutrophil cytoplasmic autoantibody (ANCA)-associated vasculitides (AAV), including MPO granulomatosis with polyangiitis (Wegener's granulomatosis), eosinophilic granulomatosis with polyangiitis (EGPA; formerly called Churg-Strauss syndrome), microscopic polyangiitis (MPA) and various forms of the disease in various organs, like the kidney and the lung.

### FIELD OF THE INVENTION

The invention is related to hydrogen sulfide (H₂S) releaser compounds and compositions for use in the treatment of antineutrophil cytoplasmic autoantibody (ANCA)-associated vasculitides (AAV), including MPO granulomatosis with polyangiitis (Wegener's granulomatosis), eosinophilic granulomatosis with polyangiitis (EGPA; formerly called Churg-Strauss syndrome), microscopic polyangiitis (MPA) and renal limited vasculitis (kidney-limited microscopic polyangiitis or kidney-limited necrotizing crescentic glomerulonephritis, NCGN).

### BACKGROUND ART

The antineutrophil cytoplasmic autoantibody (ANCA)-associated vasculitides (AAV) are a group of rare autoimmune diseases characterized by severe systemic vasculitis and the presence of autoantibodies against circulating autoantigens among which the most important are circulating anti-proteinase 3 (PR3) or anti-myeloperoxidase (MPO) autoantibodies. AAV subtypes include granulomatosis with polyangiitis (previously known as Wegener's granulomatosis), eosinophilic granulomatosis with polyangiitis (EGPA; formerly called Churg-Strauss syndrome), microscopic polyangiitis and renal limited vasculitis that all can be associated with PR3 or myeloperoxidase MPO [Jennette, J. C. and P. H. Nachman, ANCA Glomerulonephritis and Vasculitis. Clin J Am Soc Nephrol, 2017. 12(10): p. 1680-1691.; Kallenberg, C. G., Pathophysiology of ANCA-associated small vessel vasculitis. Curr Rheumatol Rep, 2010. 12(6): p. 399-405.].

Granulomatosis with polyangiitis (GPA, Wegener's granulomatosis) is characterized by granulomas formed in the airways, focal glomerulonephritis and necrotizing systemic vasculitis.

In a rare variant, microscopic polyangiitis (MPA), the symptoms of necrotizing vasculitis can be detected without immune deposits.

Churg-Strauss syndrome (also known as Eosinophilic Granulomatosis with Polyangiitis, EGPA) is a vasculitis related to eosinophil granulocytes, also characterized by formation of granuloma, often accompanied by asthmatic symptoms.

In AAV, among vital organs, the kidney was found to be the most generally affected, and the severity of the renal symptoms indicated the outcome. Thus, renal limited vasculitis (or microscopic polyangiitis limited to kidney or renal-limited necrotizing crescentic glomerulonephritis, NCGN) is particularly important variant of the disease in terms of public health. [Galesic K, Ljubanovic D, Horvatic I. Treatment of renal manifestations of ANCA-associated vasculitis. J Nephropathol. 2013 January; 2(1):6-19. doi: 10.5812/nephropathol.8971. Epub Jan. 1, 2013. PMID: 24475421; PMCID: PMC3886180.]

In patients with renal AAV the death rate is 2.7 times higher than in the average population. In the first year after occurrence of the disease the main cause of death was infection (48%) and the active vasculitis (19%), whereas in later stages cardiovascular diseases (26%), malignant transformations (22%) and infections (20%) lead the list. [88, 89][Tan, J. A., et al., Mortality in ANCA-associated vasculitis: a meta—analysis of observational studies. Ann Rheum Dis, 2017. 76(9): p. 1566-1574. Flossmann, O., et al., Long—term patient survival in ANCA-associated vasculitis. Ann Rheum Dis, 2011. 70(3): p. 488-94.]

The autoimmune nature of the disease implies that vast majority of the AAV patients were found to be positive for at least one of the ANCA autoantigens. A grouping of AAV diseases is based on the type of autoantigen, e.g. there are PR3 positive, MPO-positive and ANCA-negative vasculitides [Kitching, A. R., et al., ANCA-associated vasculitis. Nat Rev Dis Primers, 2020. 6(1): p. 71.].

MPO, i.e. myeloperoxidase, is one of the most abundant autoantigen in AAV. According to Galesic, K. et al., the prevalence of MPO in Wegener's granulomatosis is 20%, in microscopic polyangiitis 50%, in Churg-Strauss syndrome 60%, whereas in kidney-limited microscopic polyangiitis it is 70%, whereas the prevalence of ANCA-negative variants of the same diseases are 5%, 10%, 30% and 10%, respectively and the PR3 prevalence is also very different [Galesic, K. et al., 2013, infra].

The role of hydrogen sulfide is highly controversial in autoimmune inflammatory diseases. Among the widespread biological functions of hydrogen sulfide, diverse protective functions in inflammatory processes were proposed to this small endogenous mediator [Whiteman, M. and P. G. Winyard, Hydrogen sulfide and inflammation: the good, the bad, the ugly and the promising. Expert Rev Clin Pharmacol, 2011. 4(1): p. 13-32.]. Interactions with heme proteins may represent a key mechanism in these processes [Palinkas, Z., et al., Interactions of hydrogen sulfide with myeloperoxidase. Br J Pharmacol, 2015. 172(6): p. 1516-32.].

However, H₂S may easily turn out to be toxic, also depending on conditions and concentration. H₂S toxicity is caused by the reaction of H₂S with trivalent iron in oxidized cytochrome oxidase, which inhibits cellular respiration and leads to cellular hypoxia. [Gill Tamis et al. Overview on hydrogen sulfide-mediated suppression of vascular calcification and hemoglobin/heme-mediated vascular damage in atherosclerosis, Redox Biology, Volume 57, 2022, 102504, ISSN 2213-2317.]

Interactions of H₂S with MPO enzyme has been investigated and a reversible inhibitory effect of the former on peroxidase and chlorination activities of MPO was found, however, kinetic experiments have shown nevertheless that interactions between H₂S and MPO are very complex and H₂S may also function as a substrate for MPO. [Palinkas, Z., et al., Interactions of hydrogen sulfide with myeloperoxidase. Br J Pharmacol, 2015. 172(6): p. 1516-32.][Garai, D., et al., Mechanisms of myeloperoxidase catalyzed oxidation of H₂S by H₂O₂ or O₂ to produce potent protein Cys-polysulfide-inducing species. Free Radic Biol Med, 2017. 113: p. 551-563].

Thus, this Janus-faced feature of H₂S places a huge burden on the investigator to arrive at a correct conclusion, whether H₂S is beneficial or not in a specific disease and complex experimentation is required the outcome or result of which could not be predicted.

In fact, while the present inventors have also found that sulfide and sulfide donors may efficiently inhibit the formation of MPO oxidants in the supernatants of PMA activated neutrophils [Garai, D., et al. Measurements for Sulfide-Mediated Inhibition of Myeloperoxidase Activity. In: Beltowski, J. (eds) Vascular Effects of Hydrogen Sulfide. Methods in Molecular Biology, 2019, vol 2007. Humana, New York, NY. https://doi.org/10.1007/978-1-4939-9528-8_14], MPO is an autoantigen in ANCA vasculitis, and activity thereof is irrelevant from the point of view of the etiology of the AAV. In fact, presently, the persistence of MPO positivity, an increase in MPO levels or even a change in from MPO-negativity to MPO positive serotype are at most modestly predictive of future disease relapse and according to the KDIGO 2021 Clinical Practice Guideline for the Management of Glomerular Diseases, should not be used to guide treatment decisions. [KDIGO 2021 Clinical Practice Guideline for the Management of Glomerular Diseases Kidney International, (2021) 100(45) Supplement, pages S1 to S276].

The prior art results suggest that while H₂S may influence pathophysiological processes of neutrophils due to physiological effects significant in inflammation, however, discovery of the complex network of effects indicated that the overall effect is uncertain and unpredictable.

Currently there is no data regarding the role of MPO activity being predictive in the treatment of an autoimmune disease, like AAV.

Moreover, there is clear evidence that neutrophil functions were not affected by hydrogen sulfide donors in case of PMA, LPS or Escherichia coli activation.

For example, treatment with the hydrogen sulfide donor GYY4137 did not inhibit PMA or LPS induced oxidative burst. In fact, pretreatment of neutrophils with 200 μM GYY4137 before PMA activation increased the production of ROS compared to non-treated samples. Treatment with GYY4137 also inhibited apoptosis and promoted cell-viability in LPS induced neutrophils. It was demonstrated that sulfide moderately decreased the formation of ROS in LPS induced neutrophils, however these effects were measured after 17-18 h incubation. [Petrushanko, I. Y., et al. Influence of the Donor of Hydrogen Sulfide GYY4137 on the Activation of Human Neutrophils by E. coli Lipopolysaccharides. Mol Biol 53, 79-86 (2019)]

Furthermore, it was shown by another research group that various sulfide releasing compounds, such as sodium sulfide, diallyl-disulfide, diallyl-trisulfide and cysteine also inhibited neutrophil apoptosis upon PMA and bacterial activation and promoted oxidative burst. [Farahat S. et al. Effect of Hydrogen Sulfide on Essential Functions of Polymorphonuclear Leukocytes. Toxins. 2023; 15(3):198.].

Rinaldi, L. et al. even demonstrated that in the presence of NaH₂S (1.83 mM) neutrophils were more efficient in killing bacteria than in the absence of sulfide, and the sulfide donor also diminished neutrophil apoptosis. [Rinaldi, L., Gobbi, G., Pambianco, M. et al. Hydrogen sulfide prevents apoptosis of human PMN via inhibition of p38 and caspase 3. Lab Invest 86, 391-397 (2006). https://doi.org/10.1038/labinvest.3700391]

Apparently, in none of these publications was AAV, an autoimmune disease, mentioned. There is no hint in the prior art that sulfide can inhibit priming, ANCA-induced degranulation and oxidative burst. Rather, the prior art seems to suggest that hydrogen-sulfide may even increase neutrophil activation by preventing neutrophil apoptosis—a process undesired in an autimmune disease.

Actually, the treatment options of AAV diseases in the prior art are limited.

Initially, the patients usually receive immunosuppressive treatment by cyclophosphamide or rituximab, which may be augmented, depending on the status of the patient by other treatment options e.g. by glucocorticoids or methothrexate. In the KDIGO 2021 Clinical Practice Guideline for the Management of Glomerular Diseases [KDIGO 2021 infra] Recommendation 9.3.1.1 suggests that glucocorticoids in combination with cyclophosphamide or rituximab be used as initial treatment of new-onset AAV (1B).

In order to maintain remission, patients may be treated by azathioprine or methothrexate. However, only a small ratio of the patients may arrive at remission free of treatment with medicaments. [Chen, M. and C. G. Kallenberg, ANCA-associated vasculitides-advances in pathogenesis and treatment. Nat Rev Rheumatol, 2010. 6(11): p. 653-64.; Ozaki, S., ANCA-associated vasculitis: diagnostic and therapeutic strategy. Allergol Int, 2007. 56(2): p. 87-96.]

Unfortunately, due to a combination of immunosuppressants and steroid treatments the patients become susceptive to infections. Further accompanying conditions like hormonal disorders, malignancies of the bladder, hematopoietic tissues or the skin may arise as well as septic shock may be formed due to long-term side-effects. [Haubitz, M. Acute and Long-term Toxicity of Cyclophosphamide. 2007].

In the last 15 years newly suggested therapies have been considered.

As tumor necrosis factor α (TNFα) appears to be an important mediator in AAV diseases, in particular GPA, monoclonal antibodies against TNFα have been proposed for treatment, however, safety concern may arise. According to the review of Unizony S. and Stone, J. H. results suggest that monoclonal anti-TNF-α inhibitors have no role beyond traditional immunosuppression in newly diagnosed patients, whereas in refractory disease no firm conclusions can be drawn. [Unizony S. and Stone, J. H. Experimental therapies for vasculitis CHAPTER 42 Oxford Textbook of Vasculitis, Third edition, Oxford University Press 2014 Eds. Ball, Gene V., Fessler, Barri J. and Bridges S. Louis]

US20130331360A1, granted as U.S. Pat. No. 8,865,684B2 (National University of Singapore), now lapsed, discloses a range of slow H₂S releaser compound, including GYY4137 and their use as a vasodilator and thus propose their use in cardiovascular diseases, however, does not suggest or hint their use in vasculitis.

Vasoconstriction is not part of the pathophysiology of antineutrophil cytoplasmic autoantibody (ANCA)-associated vasculitis [Duvuru Geetha and J Ashley Jefferson. ANCA-Associated Vasculitis: Core Curriculum 2020, Am J Kidney Dis, 2020 January; 75(1):124-137]. Accordingly, treatment of the ANCA-associated vasculitis does not include any therapeutic approach related to vasodilator action and vasodilator agent [van Daalen et al. Developments in the Histopathological Classification of ANCA-Associated Glomerulonephritis. Clin J Am Soc Nephrol 2020 August 7; 15(8):1103-1111.] and [Rovin et al., Executive summary of the KDIGO 2021 Guideline for the Management of Glomerular Diseases Kidney Int. 2021 October; 100(4):753-779.].

With the accumulating evidence of ANCA-mediated neutrophil activation, neutrophil-oriented therapies also have been suggested. Therapeutic strategies for AAV may include removal of the offending autoantibodies (plasma exchange), modulation of the neutrophil FcγR signaling pathways, suppression of neutrophil extracellular trap formation, and anticytokine therapy. [Unizony S. and Stone, J. H. Experimental therapies for vasculitis CHAPTER 42 Oxford Textbook of Vasculitis, Third edition, Oxford University Press 2014 Eds. Ball, Gene V., Fessler, Barri J. and Bridges S. Louis].

It is known that neutrophil priming includes the translocation of MPO to the cell surface to serve as an autoantigen for ANCAs, which eventually induces a respiratory burst [Jennette, J. C. and R. J. Falk, Pathogenesis of antineutrophil cytoplasmic autoantibody-mediated disease. Nat Rev Rheumatol, 2014. 10(8): p. 463-73.]. MPO activity does not play a role in this process.

The inventors have surprisingly found that sulfide donors could also inhibit the translocation of ANCA antigen MPO and thereby neutrophil priming.

Moreover, the inventors have surprisingly found that sulfide donors could efficiently inhibit neutrophil degranulation upon ANCA activation as well as neutrophil activation by isolated IgG from ANCA patients.

Meanwhile, neutrophil phagocytosis and bacterial killing were not inhibited in the presence of sulfide, and sulfide and sulfide donor compounds had no effect on PMA induced activation of neutrophil granulocytes—a surprising fact indicating that sulfide is not harmful in this regard.

The prior art is silent about the use of sulfide donor molecules in the treatment for ANCA vasculitis, in particular wherein MPO serves as an autoantigen. Rather, based on prior art observations, the fact that sulfide can inhibit priming, and ANCA-induced degranulation and oxidative burst, are not obviously deduced from currently available data in the literature.

In a systematic series of experiments the present inventors have studied how sulfide could interfere with these processes and unexpectedly found that H₂S donor compounds are useful in alleviating MPO-related pathological processes in AAV whereas, surprisingly do not affect normal neutrophil activity necessary to maintain immune homeostasis. Specifically, while an increased H₂S serum level was active in MPO related disease processes, neutrophil phagocytosis and bacterial killing were not inhibited in the presence of sulfide, and sulfide and sulfide donor compounds had no effect on PMA induced activation of neutrophil granulocytes (a model of bacterial infection), or NADPH oxidase (NOX) activity. Oral administration of the compounds is particularly preferred. The sulfide donor compounds provided protection from kidney damage in a mouse model of MPO-ANCA vasculitis.

Thus, the present inventors have surprisingly found that a novel type of AAV treatment is possible in patients of MPO positive serotype by elevating H₂S serum levels, i.e. by H₂S donors. Slow H₂S releasing compounds are preferred.

### BRIEF DESCRIPTION OF THE INVENTION

1. The invention relates to a H₂S donor compound for use in the treatment of antineutrophil cytoplasmic autoantibody (ANCA)-associated vasculitis (AAV) in a mammalian patient having AAV of the myeloperoxidase-ANCA serotype (MPO-ANCA vasculitis). In particular, said AAV is a myeloperoxidase (MPO) positive AAV wherein said subject has an increased ANCA level against MPO.

In a preferred embodiment MPO level is measured in said patient wherein AAV is clinically diagnosed.

Preferably MPO level is measured by IIF and/or ELISA. Preferably MPO level is above a pre-determined threshold level.

2. Preferably, said H₂S donor compound is a slow H₂S releaser compound.

Preferably, said compound is used for
inhibiting neutrophil granulocyte activation by anti-MPO antibodies of mammalian subject having AAV (MPO-ANCA associated vasculitis)
inhibiting neutrophil degranulation in the mammalian subject having AAV,
inhibiting neutrophil priming (by translocation of ANCA antigen MPO) in the mammalian subject having AAV, and/or
preventing kidney damage/protecting the kidney in the mammalian subject having AAV.

In a particular embodiment the H₂S donor compound is useful in treatment of MPO-positive AAV in an early stage or prevention of manifestation of clinical symptoms, preferably by inhibiting neutrophil priming in the mammalian subject having AAV.

In a particular embodiment the H₂S donor compound is useful in treatment of renal limited vasculitis and/or in any kidney related forms of AAV.

Preferably said H₂S donor compound does not inhibit NOX activity, phagocytosis and bacterial killing of neutrophils. Preferably said H₂S donor compound does not inhibit pathogen induced neutrophil activity.

Preferably said H₂S donor compound is a low molecular weight compound.

3. Preferably, said AAV is selected from granulomatosis with polyangiitis (GPA) (Wegener's granulomatosis), microscopic polyangiitis (MPA), eosinophilic granulomatosis with polyangiitis (EGPA) (Churg-Strauss syndrome), renal limited vasculitis (AAV), AAV of the lung).

In a further preferred embodiment the AAV is renal limited vasculitis, preferably kidney-limited microscopic polyangiitis or kidney-limited necrotizing crescentic glomerulonephritis, NCGN.

In a further preferred embodiment the AAV is manifested in the lung.

In a further particularly preferred embodiment the AAV is manifested in the kidney.

In a further preferred embodiment the AAV is manifested at least in the lung and in the kidney.

In a highly preferred embodiment the compound is for use in microscopic polyangiitis (MPA), eosinophilic granulomatosis with polyangiitis (EGPA) (Churg-Strauss syndrome), or renal limited vasculitis, preferably kidney-limited microscopic polyangiitis or kidney-limited necrotizing crescentic glomerulonephritis, NCGN, highly preferably in EGPA and/or in renal limited vasculitis

4. Preferably said compound increases H₂S level in the serum of a mammalian subject after administration of said compound to said subject, preferably said subject having increased H₂S serum level after a time point of 1 h to 24 h after administration of said compound, if compared with normal H₂S serum level (or the serum level of H₂S without (or before) administration of said compound).

Normal level includes a pre-determined threshold level or range measured and calculated in control subjects without administration of said compound or the control serum level of H₂S measured in a control subject without (or before) administration of said compound.

Preferably said increased H₂S serum level is significantly higher, if measured by any relevant statistical method than normal serum level.

Preferably the H₂S serum level is meant to represent bioavailable sulfide (H₂S) levels throughout this document measured by the protocol of Ditroi et al. [Ditroi, T., et al., Comprehensive analysis of how experimental parameters affect H₂S measurements by the monobromobimane method. Free Radic Biol Med, 2019. 136: p. 146-158.]. Preferably said increased H₂S serum level is at least 1.5 times, highly preferably at least 2 times, even more preferably at least 3 times higher at specific time points than the normal H₂S serum level as measured by the method of Ditroi et al.

Preferably the compound of the invention is a H₂S releaser, preferably a slow H₂S releaser, which significantly increases bioavailable sulfide (H₂S) levels in the serum of a mammalian subject after administration of said compound to said subject after a time point of 1 h to 24 h after administration of said compound, if compared with normal H₂S serum level as control.

Preferably the H₂S donor compound of the invention, in particular the slow H₂S releaser compound is a low molecular weight compound.

5. In a particularly preferred embodiment said compound is administered orally to said mammalian patient.

Particularly preferably the H₂S donor compound decomposes and H₂S is released in the gastrointestinal tract, preferably in the stomach, or in conditions modeling the gastrointestinal tract, or the stomach.

6. In a general embodiment said compound has general formula (Y)

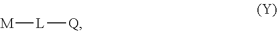

wherein
Q is a H₂S releasing moiety which, once present in the body of the mammalian patient, upon metabolism, releases H₂S into the blood stream of said mammalian patient,
   preferably
Q comprises a dithiole group (—S—S—), or
Q comprises a phosphinodithionate group (═P(S)S—)
L is an organic linking moiety,
M is a moiety which is covalently bound the rest of the molecule by a hydrolysable bond, wherein upon hydrolysis and upon metabolism and H₂S release, M is converted to a compound which is tolerable by the mammalian patient, preferably beneficial to the mammalian patient,
wherein either L or M or none of them may be missing.

In an embodiment the compound is decomposed and releases H₂S under conditions which model the gastrointestinal system of the mammalian subject, preferably under gastric model conditions.

7. In a preferred embodiment said compound has general formula (X)

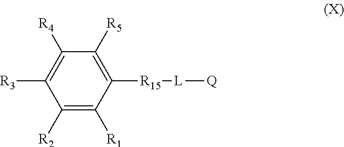

wherein
Q is a H₂S releasing moiety which, once present in the body of the mammalian patient, upon metabolism, releases H₂S into the blood stream of said mammalian patient,
   in a preferred embodiment
   Q comprises a 5 to 6 membered heterocycle comprising a dithiole group (—S—S—), or
   Q comprises a phosphinodithionate group (═P(S)S—)
L is a linking moiety,
   in a preferred embodiment L is C1-C8 alkylene, (preferably methylene), —, —O—, S, —NH—, an aryl, a C1-C4 alkylaryl, a 5 to 6 membered heterocycle, optionally linked with Q via a C₁-C₄ alkylene,
wherein at least two, preferably three or four of R₁, R₂, R₃, R₄ and R₅ is H,
and R₁, R₂, R₃, R4 and R₅ are, independently, selected from
   H, halogen, pseudohalogen, —CN, —OH, —SH, —NO₂, —NH₂, —NHCH₃, —COOH, CONH₂,
   substituted or unsubstituted C1-C8 alkyl, C2-C8 alkenyl, C2-C8 alkynyl, C1-C8 alkoxy, C1-C8 alkylamide, C6-C10 aryl, C7-C12 alkylaryl (aralkyl), 5 to 10 membered heteroaryl, 6-12 membered alkyl-heteroaryl, C1-C5 amide a C1-C8 carbonyl, (preferably a C2-C8 alkylcarbonyl, C3-C8 alkenylcarbonyl, C3-C8 alkynylcarbonyl,) a C1-C8 carboxyl, (preferably a C2-C8 alkylcarboxyl, C3-C8 alkenylcarboxyl or C3-C8 alkynylcarboxyl), a C2-C8 carboxylate ester (preferably a C2-C8 alkylester, C3-C8 alkenylester or C3-C8 alkynylester), said substituent, said substituent, if any, being selected from halogenide, pseudohalogenide, —OH, —SH, —OMe, —NO₂, —NH₂, —NHMe,
   —OCOR₁₆, —COOR₁₇, —OR₁₈, —CONHR₁₉, wherein R₁₆, R₁₇ R₁₈ and R₁₉ are selected from H and substituted or unsubstituted C1-C8 alkyl, C2-C8 alkenyl, C2-C8 alkynyl, C1-C8 alkoxy, C1-C8 alkylamide, C6-C10 aryl, C7-C12 alkylaryl (aralkyl), 5 to 10 membered heteroaryl, 6-12 membered alkyl-heteroaryl, C1-C5 amide a C1-C8 carbonyl, (preferably a C2-C8 alkylcarbonyl, C3-C8 alkenylcarbonyl, C3-C8 alkynylcarbonyl,) a C1-C8 carboxyl, (preferably a C2-C8 alkylcarboxyl, C3-C8 alkenylcarboxyl or C3-C8 alkynylcarboxyl), a C2-C8 carboxylate ester (preferably a C2-C8 alkylester, C3-C8 alkenylester or C3-C8 alkynylester), said substituent, said substituent, if any, being selected from halogenide, pseudohalogenide, —OH, —SH, —Ome, —NO₂, —NH₂, —NHMe,
   preferably R₁ and R₃ being independently selected from —OCOR₁₆, —COOR₁₇, —OR₁₈, wherein R₁₆, R₁₇ and R₁₈ are selected from H and substituted or unsubstituted C1-C8 alkyl, (preferably methyl or ethlyl) C2-C8 alkenyl, C2-C8 alkynyl, a C1-C8 carbonyl, (preferably a C2-C8 alkylcarbonyl, C3-C8 alkenylcarbonyl, C3-C8 alkynylcarbonyl,) a C1-C8 carboxyl, (preferably a C2-C8 alkylcarboxyl, C3-C8 alkenylcarboxyl or C3-C8 alkynylcarboxyl), a C2-C8 carboxylate ester (preferably a C2-C8 alkylester, C3-C8 alkenylester or C3-C8 alkynylester), said substituent, said substituent, if any, being selected from halogenide, pseudohalogenide, —OH, —SH, —Ome, —NO₂, —NH₂, —NHMe,
   more preferably
   wherein R₂, R₃ and R₅ are H,
R₁₅ is selected from C1-C8 alkylene, C1-C8 alkylether, C1-C8 carboxylate, preferably —(CH₂)ₙ—(CO)O—wherein n is 0, 1, 2 or 3, preferably 0 or 1,
or a pharmaceutically acceptable salt and/or solvate and/or complex thereof.

8. In a preferred embodiment said compound has general formula I

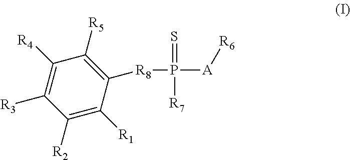

wherein
R₇ is selected from
   a 3 to 10 membered, preferably 5 to 10 membered, more preferably a 5 to 6 membered heterocycle
   —NH—R₂₃ wherein R₂₃ is selected from C1-4 alkyl, C1-4 alkycarbonyl,
R₈ (preferably wherein R₈ is L) is selected from
   —NH—
   —CH₂—, —O—, S or NH, preferably CH₂, O or NH,
   or the meaning of R₁₈ is nothing (GYY, Lawesson's reagent, JK donors),
A is selected from
   —S⁻, wherein if A is —S⁻, then R₆ is nothing,
   —S— and —S⁻, wherein if A is —S— then
      R₆ may be selected from H, a 5 to 10 membered, preferably a 6 membered, optionally substituted heterocycle, C1-4 alkyl or C6-C10 aryl, or
      A is —S—, and R₆ and R₇ together form a moiety having the formula

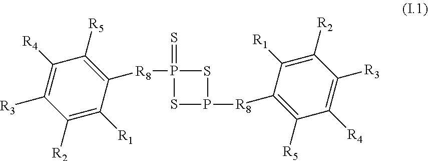

wherein R₁ to R₅ and R₈ are, independently, as defined above, wherein substituents of the two rings are either the same or different,
—O⁻
—O—, wherein if A is —O— then
   R₆ may be selected from H, a 5 to 10 membered, preferably a 6 membered heterocycle, C1-4 alkyl or C6-C10 aryl, or
   a 10 to 20 membered organic moiety having one or two 5 to 6 membered heterocycles and optionally at least one 1 to 8 membered open chain moiety, optionally comprising 1 to 4, preferably 1 to 3 heteroatoms, and/or
   R₆ is an organic moiety which, once hydrolysed, converted to a compound which is tolerable by the mammalian patient, preferably beneficial to the mammalian patient,
R₁, R₂, R₃, R₄ and R₅ are as defined above, or preferably R₁, R₂, R₃, R₄ and R₅ are, independently, selected from H, halogen, —CN, —OH, —SH, —NO₂, —NH₂, —NHCH₃, —COOH, CONH₂, wherein at least two of R₁, R₂, R₃, R₄ and R₅ is H;
preferably R₃ is —OCH₃ or each of R₁, R₂, R₃, R₄ and R₅ is H,
or a pharmaceutically acceptable salt and/or solvate and/or complex thereof,
in a particular embodiment a dichloromethane complex and/or a morpholinium salt.

9. In a particular embodiment said compound has general formula I.2

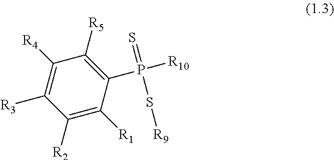

wherein
R₉ is H, wherein said H may be dissociated the —S—R₉ is —S⁻,
A is selected from nothing, and —O— and
wherein A is —O— then R₆ is
   a 10 to 20 membered organic moiety having one or two 5 to 6 membered heterocycles and optionally at least one 1 to 8 membered open chain moiety, optionally comprising 1 to 4, preferably 1 to 3 heteroatoms, or
wherein A is nothing then R₆ is
   a 5 to 10 membered, preferably a 6 membered heterocycle, preferably an O and/or N-containing heterocycle, in particular a morpholino group connected via N,
R₁, R₂, R₃, R₄ and R₅, is selected from H, C1-4 alkyl, C1-4 alkoxy, halogenide, wherein at least two of R₁, R₂, R₃, R₄ and R₅ is H; preferably R₃ is —Ome or each of R₁, R₂, R₃, R₄ and R₅ is H, or a pharmaceutically acceptable salt and/or solvate and/or complex thereof;

10. In a further particular embodiment said compound has general formula I.3

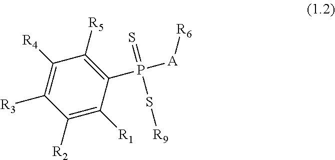

wherein R₁₀ is a 5 to 10 membered, preferably a 6 membered heterocycle, preferably an O and/or N-containing heterocycle, in particular a morpholino group connected via N,
R₁, R₂, R₃, R₃ and R₅ as well as R₉ are as defined above.
wherein particularly preferably said compound has general formula I.3.1

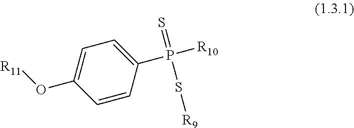

wherein R₁₁ is a C1-4 alkyl,
wherein preferably the compound for use according to claim 9 is GYY4137.

11. In a further said compound has general formula I.4

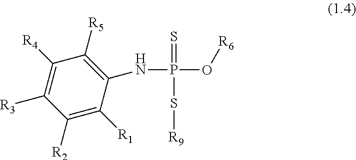

wherein R₁, R₂, R₃, R₃ and R₅ are as defined above, preferably each of them are H,
R₆ and R₉ are as defined as above.

12. In an alternative embodiment said compound having general formula II (preferably, if dependent of claim 6 or 7, as moiety M),

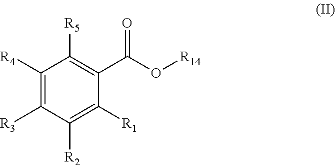

wherein R₁, R₂, R₃, R₄ and R₅ are as defined above (in claim 7)
wherein at least two, preferably three or four of R₁, R₂, R₃, R₄ and R₅ is H,
wherein preferably R₁, R₂, R₃, R₄ and R₅ are, independently, selected from the group consisting of, (preferably R₁ is selected from the group consisting of
   H, halogen, pseudohalogen, —CN, —OH, —SH, —NO₂, —NH₂, —NHCH₃, —COOH, CONH₂, preferably OH, and
   —OCOR₁₆, —COOR₁₇, —OR₁₈, wherein R₁₆, R₁₇ R₁₈ and R₁₉ are selected from H and substituted or unsubstituted C1-C8 alkyl, C2-C8 alkenyl, C2-C8 alkynyl, C1-C8 alkoxy, C1-C8 alkylamide, C6-C10 aryl, C7-C12 alkylaryl (aralkyl), 5 to 10 membered heteroaryl, 6-12 membered alkyl-heteroaryl, C1-C5 amide a C1-C8 carbonyl, (preferably a C2-C8 alkylcarbonyl, C3-C8 alkenylcarbonyl, C3-C8 alkynylcarbonyl,) a C1-C8 carboxyl, (preferably a C2-C8 alkylcarboxyl, C3-C8 alkenylcarboxyl or C3-C8 alkynylcarboxyl), a C2-C8 carboxylate ester (preferably a C2-C8 alkylester, C3-C8 alkenylester or C3-C8 alkynylester), said substituent, said substituent, if any, being selected from halogenide, pseudohalogenide, —OH, —SH, —Ome, —NO₂, ONO₂, —NH₂, —NHMe,
      preferably R₁ being selected from —OCOR₁₆, —COOR₁₇, —OR₁₈, wherein R₁₆, R₁₇ and R₁₈ are selected from H and substituted or unsubstituted C1-C8 alkyl, (preferably methyl or ethlyl) C2-C8 alkenyl, C2-C8 alkynyl, a C1-C8 carbonyl, (preferably a C2-C8 alkylcarbonyl, C3-C8 alkenylcarbonyl, C3-C8 alkynylcarbonyl,) a C1-C8 carboxyl, (preferably a C2-C8 alkylcarboxyl, C3-C8 alkenylcarboxyl or C3-C8 alkynylcarboxyl), a C2-C8 carboxylate ester (preferably a C2-C8 alkylester, C3-C8 alkenylester or C3-C8 alkynylester), said substituent, said substituent, if any, being selected from halogenide, pseudohalogenide, —OH, —SH, —Ome, —NO₂, ONO₂, —NH₂, —NHMe,
      more preferably R₂, R₃, R₄ and R₅ are H, and R₁ is as define above, or
         even more preferably R₁ is selected from —OH, —OCOR₁₆, wherein R₁₆ is selected from H and substituted or unsubstituted C1-C8 alkyl, (preferably methyl or ethlyl) C2-C8 alkenyl, C2-C8 carboxylate ester (preferably a C2-C8 alkylester, C3-C8 alkenylester or C3-C8 alkynylester), said substituent, said substituent, if any, being selected from halogenide, pseudohalogenide, —OH, —SH, —Ome, —NO₂, ONO₂, —NH₂, —NHMe,
R₁₄ is a group having formula III.2

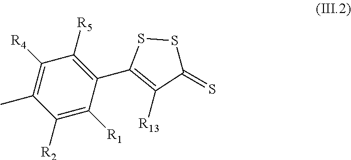

wherein in formula III.2
R₁, R₂, R₄ and R₅ are, independently, selected from H, halogen, pseudohalogen, —CN, —OH, —SH, —NO₂, —NH₂, —NHCH₃, —COOH, CONH₂, substituted or unsubstituted C1-C8 alkyl, C2-C8 alkenyl, C2-C8 alkynyl, C1-C8 alkoxy,
   preferably H or OCH₃,
R₁₃ is selected from H and OCH₃, preferably

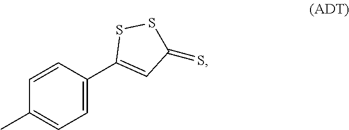

wherein in a preferred embodiment the compound is ADT-OH

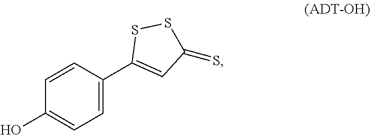 [SMILES: Oc1ccc(-c2cc(=S)ss2)cc1]

13. Preferably, said compound being selected from

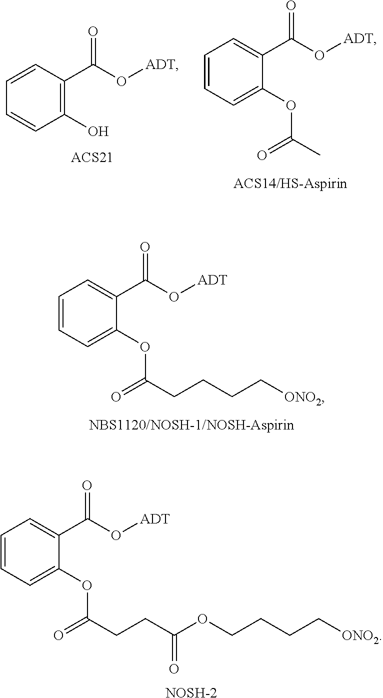

14. In a further alternative embodiment the H₂S donor compound is a natural H₂S donor compound selected from allicin, alliin, diallil-trisulfide, diallil disulfide, diallil tetrasulfide, ajoene (e.g. E-ajoene or Z-ajoene), anthionine, ovothiol, S-Allylmercaptocysteine (SAMC), 3H-1,2-Dithiole-3-thione, alpha lipoic acid.

15. In a further alternative embodiment the H₂S donor compound comprises a substituted or unsubstituted, 5 membered heterocycle comprising a dithiole group (—S—S—, preferably an 1,2 dithiolane group or an 1,2 dithiolene group, preferably a group having the formula 5:

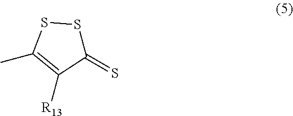

wherein R₃ is H or a C1-8 alkyl, preferably wherein

16. In a preferred embodiment the H₂S donor compound for use according to any of paragraphs 1 to 15 wherein said composition is administered daily, preferably one, two or three times a day, for at least one month, two months, three months, half year, one year or more.

### Pharmaceutical Compositions (Medicaments)

17. The invention also relates to a pharmaceutical composition comprising a H₂S donor compound for use according to any of paragraphs 1 to 15, preferably according to any of paragraphs 5 to 15, said composition being formulated for systemic administration.

18. The pharmaceutical composition preferably comprises a H₂S donor compound for use according to claim 17,
said composition being formulated for oral administration, preferably in the form of capsules and/or tablets wherein said composition is protected from light, moisture and decay to form H₂S during storage.

19. The pharmaceutical composition preferably comprises a H₂S donor compound for use according to claim 17, said composition being formulated for intravenous administration.

20. The invention also relates to a pharmaceutical composition comprising a H₂S donor compound for use according to claim 19,
said composition being formulated in the form of a powder which can be reconstituted in
an intravenous injection or infusion,
intraperitoneal injection or infusion or
infusion,
with an appropriate solution (e.g. Ringer-solution, Ringer-lactate solution, buffered solutions etc.).

21. The pharmaceutical composition preferably comprises a H₂S donor compound for use according to claim 17, said composition being formulated as drops, e.g.
eye drops
intranasal drops.

22. The invention also relates a pharmaceutical composition comprising a H₂S donor compound for use according to any of paragraphs 1 to 15, preferably according to any of paragraphs 5 to 15,
said composition being formulated for topical administration,
preferably in the form of ointment, gel or emulsion,
said topical administration form comprising excipients to protect the H₂S donor compound moisture and/or decay to form H₂S during storage and optionally from light.

23. Composition according to paragraphs 17 22 are formulated, including packaged and stored as desired to obtain an appropriate shelf-life.

The pharmaceutical composition or formulation according to any of paragraphs 17 to 22, said composition comprising a light-protective package.

24. In a preferred embodiment before treatment the AAV patients are diagnosed for MPO-ANCA positivity.

AAV patients are patients who are considered to show AAV symptoms according to any guidelines as described herein (in a particular embodiment (the 2012 revised International Chapel Hill Consensus Conference nomenclature of vasculitides, [Jennette J C, 2012, infra], or any other diagnostic or rating tool, e.g. Birmingham Vasculitis Activity Score (BVAS) and the Vasculitis Damage Index (VDI) or as referred in [Ball, Gene V., Fessler, Barri J. and Bridges S. Louis, Eds., Oxford Textbook of Vasculitis, Third edition, Oxford University Press 2014] or known in the art or known at the time given. In this regard the level of diagnosis which suggests that the patient may have or may develop AAV is sufficient provided that such level advises (or warrant) to
start treatment,
carry out further diagnostic step (e.g. biopsy) and/or
detect ANCA.

In this regard the methods described or cited in Chapter “Diagnosis of MPO-ANCA associated vasculitis patients” are specifically contemplated or, if appropriate”, incorporated herein by reference.

The level “advises to” included a case when symptoms from at least two organs refer to AAV, or when the protocol in force advises start of treatment or further investigation.

In a particular embodiment the mammalian patient has higher ANCA titer and multiple affected organ systems (as defined by Houben E et al. 2016, infra).

25. The invention also relates to a method of treatment wherein said composition or formulation is administered to a mammalian patient of the MPO-ANCA serotype as defined herein in order to prevent or treat an MPO associated AAV in said patient.

In a particular embodiment the invention relates to a treatment method for the treatment of a condition as defined in any of paragraphs 1 to 24.

In a particular embodiment oral administration is applied.

In a particular embodiment diagnostic test is carried out, as disclosed herein to identify the patient group to be treated.

### Definitions

A “subject” as used herein is an individual of an animal species, preferably a vertebrate, more preferably a mammalian or avian species, in particular a mammalian species, highly preferably the individual is a primate, a hominid or a human. A “patient” is a subject who is or intended to be under medical or veterinarian care, observation, supervision, diagnosis or treatment.

A “treatment” of a subject refers to any process, action, therapy, or the like, wherein the subject or patient is under aid, in particular medical or veterinarian aid with the object of improving the subject's or patient's condition, either directly or indirectly. Improving the subject's condition may include restoring or maintaining normal function of an organ or tissue, preferably at least partly restoring or maintaining health (medical or veterinarian treatment). Treatment typically refers to the administration of an effective amount of a compound or composition described herein. Preferably the administration of the compound is oral administration and results in an appropriate serum level of H₂S in the patient. Particularly preferably treatment includes both medical or veterinarian treatment in the early phase of the disease e.g. in the phase of neutrophil priming in an MPO-ANCA positive patient, in particular AAV patient.

In a broader sense treatment includes both medical or veterinarian treatment in the early phase of the disease including prevention (or prophylaxis) i.e. prevention of the onset of a disease symptoms or prevention of manifestation of the disease in an MPO-ANCA positive patient. In a more limited sense prevention is not covered.

“Disease activity” of ANCA-associated vasculitis represents signs or symptoms attributable to active disease in any organ system

“Remission” is defined as the absence of manifestations of vasculitis. Remission may be understood as the remission of clinical symptoms of the disease whereas MPO positivity remains.

“Relapse” is defined as the occurrence of increased disease activity after a period of partial or complete remission.

A “pharmaceutical composition” of the invention is a composition of matter which comprises at least one H₂S donor compound of the invention for use in MPO-ANCA positive patient, said composition comprising an active agent and at least one further substance. Preferably the compound of the invention is present in an effective amount, preferably in an amount resulting in an effective serum level of H₂S in a mammalian subject. Compositions may also comprise further biologically active substances useful e.g. in a combination therapy. Furthermore, the compositions may comprise biologically acceptable carriers, formulation agents, excipients etc. which may be known in the art.

The term “effective amount” qualifies the amount of a compound required to exert the effect of the active agent in a composition. A “therapeutically effective amount” is sufficient to relieve the AAV patient or prevent (or prevent occurrence or manifestation of AAV symptom or exacerbation of the disease condition) one or more of the symptoms or characteristic parameters of a condition, e.g. a disorder or disease.

A “H₂S donor compound” is a compound, that degrades in response to an environmental stimulus (for example water, light, a nucleophile such as a thiol, the action of an enzyme, or other stimuli) to release H₂S.

Preferably the compound of the invention, once administered in an effective amount, results in an effective serum level of H₂S in a mammalian subject. Preferably the “H₂S donor compound” has a “sulfide releaser moiety” used herein as a sulfur-containing part of the compound which is the part of the molecule responsible for the release of H₂S in the patient's body, preferably resulting in the elevation of H₂S level in the patient's serum. In particular a H₂S donor compound is a compound as defined by Powell, Chadwick R. et al., [in: Powell, Chadwick R., Kearsley M. Dillon, and John B. Matson. “A review of hydrogen sulfide (H₂S) donors: Chemistry and potential therapeutic applications.” Biochemical pharmacology 149 (2018): 110-123]. In a particular aspect the H₂S is released under biological conditions e.g. in a subject or in a condition providing a model of biological conditions, including an in vitro model.

A “slow H₂S releaser” compound is a H₂S donor compound from which H₂S is released slower, preferably for a 10 times longer time, more preferably for a 100 times longer time than from a reference sulfide compound, preferably from Na₂S under appropriate reference conditions e.g. under identical or appropriately comparable conditions. An example for the measurement of the time period of the release of hydrogen sulfide from Na₂S in a cellular environment is indicated by Vitvitsky et al. on FIG. 2/A [Vitvitsky, Victor, et al. “The mitochondrial NADH pool is involved in hydrogen sulfide signaling and stimulation of aerobic glycolysis.” Journal of Biological Chemistry 296 (2021)] or [Lee Z W et al. The slow-releasing hydrogen sulfide donor, GYY4137, exhibits novel anti-cancer effects in vitro and in vivo. PLoS One. 2011; 6(6): e21077. doi: 10.1371/journal.pone.0021077]. Other methods of measurement are known in the art.

Terms “sulfide” and “H₂S” in particular bioavailable sulfide and H₂S are used interchangeably and include all protonation isomers (in particular H₂S, HS⁻, S₂⁻), unless otherwise indicated.

A “low molecular weight compound” as defined herein is a compound having a molar weight a molar weight or molecular weight (used interchangeably herein) lower than 2000 Da or 1500 Da, in particular lower than 1000 Da, preferably 900 Da. Preferably the low molecular weight compound is an inorganic, metalloorganic or organic compound. In an embodiment the compound is different from a polymeric molecule of biological origin (particularly a biological molecule isolated from nature), like a peptide or nucleic acid; in a particular embodiment a synthetic or semi-synthetic compound. A low molecular weight compound may be a small molecule or a salt, like a Li salt e.g. an organic Li salt or LiCl. In a preferred embodiment a low molecular weight compound is understood as a compound which can penetrate into the cell through the cell membrane without the use of cell surface receptors, in particular wherein it has a molecular weight lower than 1000 Da, preferably 900 Da.

A “moiety” is used herein as a part of a molecule which can be derived in principle by removing another part, even a hydrogen atom or a group or any part thereof.

An “sulfide releaser moiety” as used herein is a sulfur-containing part of the compound of the invention which is the part of the molecule which is responsible for the release of H₂S in the patient's body, preferably resulting in the elevation of H₂S level in the patient's serum.

As used herein, the term “alkyl” alone or in combinations means a straight or branched-chain (if appropriate) saturated hydrocarbon group containing preferably 1 to 15, 1 to 10 or 1 to 8 carbon atom(s) or in particular 1 to 6 or 1 to 4, 1 to 3 or 1 to 2 carbon atom(s) [i.e. “C₁₋₁₅”, “C₁₋₁₀”, “C₁₋₈”, “C₁₋₆” or in particular “C₁₋₄”, “C₁₋₃” or “C₁₋₂)” alkyl groups, respectively], such as particularly preferably methyl, ethyl, propyl or isopropyl groups.

As used herein, the term “alkoxy” means an alkyl-O— group in which the alkyl group is as previously described. The bond to the rest of the molecule or complex, i.e. the parent moiety is through the oxygen (if to a carbon atom, ether oxygen).

The term “alkoxy alkyl” means an alkyl group which is substituted by an alkoxy group, i.e. an alkyl-O— group as previously described. The bond to the alkyl moiety is through the oxygen, i.e. it is an ether oxygen.

As used herein, the terms “carbonyl”, “alkyl-carbonyl”, “alkenyl-carbonyl” and “alkynyl-carbonyl” mean a moiety having carbonyl group optionally substituted with an alkyl group, alkenyl group and alkynyl group, respectively. In a wider sense the group can be connected by either the alkyl, alkenyl or alkynyl or via the carbonyl group. In a preferred embodiment, i.e. narrower sense, the group bond to the parent moiety is through the carbon of the carbonyl group. In a preferred embodiment the “alkyl-carbonyl”, “alkenyl--carbonyl” and “alkynyl-carbonyl” is alkanoyl, alkenoyl and alkynoyl, respectively.

This definition of wider sense and narrower sense pertains to any analogous groups with a functional group used herein even if not defined separately.

As used herein, the terms “carboxyl”, “alkyl-carboxyl”, “alkenyl-carboxyl” and “alkynyl-carboxyl” are defined to mean a moiety having carboxyl group optionally substituted with an alkyl group, alkenyl group and alkynyl group, respectively, wherein bond to the parent moiety is through the carboxyl group. The group can be connected by either the alkyl, alkenyl or alkynyl or via the carboxyl group (in the latter case being an esther).

An “alkenyl” as used herein, alone or in combinations, means a straight or branched-chain unsaturated hydrocarbon group containing at least one carbon-carbon double bond, said hydrocarbon group containing preferably from 2 to 20, preferably 2 to 15, 2 to 10 or 2 to 8 carbon atoms or 2 to 6, 2 to 4, 2 to 3 or 2 carbon atoms [i.e. “C₂₋₂₀”, “C₂₋₁₅”, “C₂₋₁₀”, “C₂₋₈”, “C₂₋₆” or “C₂₋₄”, “C₂₋₃” or “C₂” alkyl groups, respectively].

An “alkynyl” as used herein is defined analogously to alkenyl mutatis mutandis.

A “heterocyclic” ring as used herein is a cyclic moiety that has, besides carbon atom(s), atoms of at least one non-carbon element as member(s) of its ring(s). A heterocycle may comprise multiple rings, e.g. it may comprise an aromatic heterocycle and, fused to the aromatic heterocycle another ring which may or may not be aromatic; i.e. if it is not aromatic it may form a cyclic substituent of the aromatic heterocycle. In a preferred embodiment, if the heteroaryl comprises multiple, in particular two fused rings, both rings are aromatic. Preferably the ring(s) of the heterocyclic moiety is/are 5 to 6 membered ring(s).

The term “heterocycloalkyl” refers to a “heterocyclic” ring which has a straight or branched-chain (if appropriate) saturated hydrocarbon group and which may have an alkyl substituent on the heterocycle, optionally a heteroaryl and/or which is derivable from cycloalkyl group as defined above, wherein at least one of the carbon atoms of the ring is replaced with a heteroatom such as, but not limited to, nitrogen or oxygen.

An “aromatic” moiety as used herein can be described as a planar cyclic moiety (a ring) wherein the single bonds (called σ-bonds) between the ring-forming atoms are formed from overlap of hybridized atomic sp2-orbitals in line between the carbon nuclei, wherein a system of delocalized π-bonds are formed from overlap of atomic p-orbitals of each of the ring forming atoms above and below the plane of the ring and wherein the number of i electrons, which is provided by the ring-forming atoms, participates in according to molecular orbital theory, must be equal to 4n+2 (Hückel's rule), in which n=1, 2, 3, etc., preferably 1 or 2, for a single ring with six it electrons, n=1. The ring-forming atoms typically provide one or two it electrons to the delocalized it electron system.

The term “heteroaryl” is defined herein as a group or molecule that contains an aromatic heterocycle, preferably a moiety that has at least one heteroatom, as “member”, incorporated within an aromatic ring. Examples of heteroatoms include nitrogen, oxygen and sulfur, preferably nitrogen and oxygen. In an embodiment a heteroaryl may comprise an aromatic heterocycle and, fused to the aromatic heterocycle another ring which may or may not be aromatic; i.e. if it is not aromatic it may form a cyclic substituent of the aromatic heterocycle. In a preferred embodiment, if the heteroaryl comprises multiple, in particular two fused rings, both rings are aromatic. Members of a heteroaryl relate to the ring-forming atoms, either carbon atom(s) or heteroatom(s).

The term “aryl” as used herein is a group that contains any carbon-based aromatic ring which is preferably a mono- or bicyclic group, wherein the bicyclic group preferably comprises two fused rings. In a preferred embodiment the aryl group consists of carbon as ring atoms, i.e. “members” only. In a broader meaning the term aryl also includes optionally “heteroaryl”. Optionally, the term “aryl” is limited to non-heteroaryl which is also included into the term aryl and defines a group that contains an aromatic group that does not contain a heteroatom. An aryl group may be substituted or unsubstituted (i.e. optionally substituted). If the aryl group is substituted it may be substituted with any substituent, and examples of the substituent include C₁₋₄ alkyl, C₂₋₄ alkenyl, C₁₋₃ alkyloxy, C₁₋₃ alkanoyl, C₁₋₃ alkylamine, C₁₋₃ alkylamide, halogen, etc.

The term “aralkyl” as used herein refers to an aryl alkyl group which is linked to the parent molecule through the alkyl group, which may be further optionally substituted with one or more, preferably one to three or one to two alkyl substituents. Thus, the aryl group may be substituted with an alkyl substituent, preferably each substituent being not larger than a C₁₋₄ alkyl.

“Aryl” or “heteroaryl” may comprise a monocyclic ring, a condensed ring, or a polycyclic ring in which a single ring is bounded by a single bond, preferably a monocyclic or bicyclic ring.

As used herein, the term “fused ring” means that the ring is fused with at least one other ring to form a group of a compound which comprises two or more rings wherein a single bond between two-member atoms of the rings is, together with said two members, common in, i.e. shared by the two rings. An example of fused rings is a polycyclic aryl. A polycyclic aryl is understood herein as a group that contains multiple rings of a carbon-based group among which at least one ring is an aryl and which optionally may also comprise a cycloalkyl and/or a heterocycloalkyl.

A “substituted” moiety comprises a substituent selected from the groups and moieties as defined herein; however, a substituent is preferably smaller, i.e. shorter, i.e. consists of not more, preferably less atoms than the moiety which is/are substituted thereby. In the present invention, “optionally substituted”, i.e. “unsubstituted or substituted” means that it may be substituted with any substituent.

In general formulae of the description H atoms are typically not shown, however, a skilled person is able to understand said formulae and recognize the full structure.

Terms expressing optionality, like optional embodiments or variants, are used throughout the specification, among which “particular” relates to embodiments or variants of particular interest and “preferred” to those which are preferred for some reason.

The singular forms “a”, “an” and “the”, or at least “a”, “an”, include plural reference unless the context clearly dictates otherwise.

The term “comprises” or “comprising” or “including” are to be construed here as having a non-exhaustive meaning and allow the addition or involvement of further elements, e.g. features or method steps or members or components to anything which comprises the listed elements. “Comprising” can be substituted by “including” if the practice of a given language variant so requires or can be limited to “consisting essentially of” if other elements than those listed are not essential to reduce the invention to practice or “consisting of” in no other elements may be present.

### Abbreviatoins

AAV: ANCA-associated vasculitides
ANCA: Antineutrophil cytoplasmic antibody
EGPA: Eosinophilic granulomatosis with polyangiitis (or Churg-Strauss syndrome).
GPA: Granulomatosis with polyangiitis (or Wegener's granulomatosis),
IgG Immunoglobuline
MPA microscopic polyangiitis
MPO: Myeloperoxidase
NCGN: Necrotizing crescentic glomerulonephritis,
NOX: NADPH oxidase
PAS: Periodic Acid-Schiff Stain
PBS: Phosphate buffered saline
PMA Phorbol 12-myristate 13-acetate
PR3: Proteinase-3
SOD: Superoxide Dismutase

### BRIEF DESCRIPTION OF THE FIGURES

FIG. 1 Effect of sulfide on neutrophil activation
(A) Effect of sodium sulfide on H₂O₂ production by PMA activated neutrophils.
The samples were treated with different concentrations of NaH₂S (0-100 μM) for 5 minutes before activation with PMA. Values obtained for the samples non-treated with sulfide were considered as 100%. H₂O₂ is produced by the NOX2 enzyme complex of the cells. H₂O₂ of cell supernatants were measured using the FOX-1 assay. The production of H₂O₂ was not affected up to 50 μM of added Na₂S.
(B) Effect of sodium sulfide on the oxidative burst of PMA-activated neutrophils. Cells were treated with different concentrations of sodium sulfide (0-50 μM) and incubated in the presence of WST-1, 5 g/ml catalase and 100 ng/ml PMA for 20 minutes. WST-1 was reduced by superoxide produced by NOX2 in the cell supernatants and it was measured spectrophotometrically at 560 nm.
(C) Effect of GYY4137 sulfide donor on the oxidative burst of PMA-activated neutrophils. Samples were prepared with the WST-1 reagent, treated with different concentrations of GYY4137 (0-100 μM) and induced with PMA for 20 minutes. Reduced WST-1 was measured from the cell supernatants at 560 nm.
(D) Effect of GYY4137 sulfide donor on the oxidative burst of ANCA-activated neutrophils were measured with the WST-1 assay. Cells were prepared with the WST-1 assay conditions and treated with GYY4137 prior (black columns) to treatment with 2 ng/ml TNF-α, or 15 minutes after starting TNF-α treatment (patterned columns). 15 minutes after the addition of TNF-α, the 300 g/ml healthy or ANCA IgG was added and incubated for 60 minutes. Reduced WST-1 was measured from the cell supernatants at 560 nm by photometry.

FIG. 2 Effect of GYY4137 on neutrophil degranulation. Neutrophil granulocytes were incubated in the presence of cytochalasin B for 10 minutes in HBSS buffer then treated with different concentrations of GYY4137 (0-100 μM) for 5 minutes. Cells were then activated with PMA or ANCAs and the (3-glucuronidase activity or the protein levels were investigated from the cell supernatants. β-glucuronidase activity was determined by measuring the g Phenolphthalein liberated in 19 hours that was measured spectrophotometrically at 520 nm. (A) Effect of GYY4137 on β-glucuronidase activity. Cells were treated with 3% Triton X in the β-glucuronidase assay conditions in the absence of GYY4137. Cell supernatants were incubated in the presence of different concentrations of GYY4137 (10-100 μM) and Phenolphthalein-glucuronide for 19 hours. (B) Effect of GYY4137 neutrophil degranulation of PMA-activated cells. Cells were prepared with the β-glucuronidase assay conditions and treated with GYY4137 an described above. 100 ng/ml PMA was added to the samples and incubated for 30 minutes. Phenolphthalein that was liberated by β-glucuronidase in supernatants was measured spectrophotometrically at 520 nm. (C) Cells were prepared with the β-glucuronidase assay conditions, treated with GYY4137 and activated by PMA. SDS-PAGE and silver staining were performed using 40 μl of each supernatants. (D) Cells were prepared with the β-glucuronidase assay conditions and treated with GYY4137 sulfide donor (0-100 μM) as described above. Cells were treated with TNF-alpha, then 300 g/ml ANCA IgG was added to the samples and incubated for 30 minutes. Phenolphthalein that was liberated by β-glucuronidase in supernatants was measured spectrophotometrically at 520 nm. On the figure 0% inhibition marks values from sample non-treated with sulfide donor.

FIG. 3 Effect of sulfide on the translocation of MPO onto the cell surface. In the presence of 10 ng/ml TNF-α neutrophils translocate MPO to the cell surface. In this experiment neutrophils were incubated for 30 minutes on gelatine (0.2%) treated glass coverslips in 24-well plates to allow them to adhere. GYY4137 (25 jM or 50 μM) or DPBS (Dulbecco's phosphate buffer) were added 5 minutes or at the same time with TNF-α treatment (added after TNF-α). The order of inscriptions shows the order of treatment on the figures. The cells were incubated for 30 minutes after the addition of TNF-α then fixed with 4% PFA. Surface MPO (second column) was labeled before permeabilization with goat anti-MPO antibody then Alexafluor 488 rabbit anti-goat secondary antibody. After permeabilization with 1% Triton X granular MPO (third column) was labeled with goat anti-MPO antibody then Alexa fluor 568 rabbit anti-goat secondary antibody and the nucleus (first column) was labeled with Hoechst 33258. After labeling the cells were mounted with Prolong™ Glass Antifade Mountant on glass slides and observed with STED microscopy. On (A) scale allows to see more neutrophils whereas on (B) in a single cell granular MPO within the cell (column 3) and cell surface MPO located at one side (polar arrangement) can be observed due to different magnification (scale).

FIG. 4 Sulfide's effect on neutrophil phagocytosis and bacterial killing (A) Neutrophils at 4*10⁴ cells/well were incubated with Fluorescent-labeled Escherichia coli K-12 BioParticles in the presence or absence of different concentrations of GYY4137 (10-100 μM) in EZ Slide glass chamber slides for 2 hours. After incubation the wells were washed then observed with fluorescent microscopy. Cytochalasin D phagocytosis inhibitor was used to provide negative control. (B) Plate assay to determine the effect of sulfide on neutrophil phagocytosis. Neutrophils 3*10⁴ cells/well were prepared and treated with GYY4137 and bioparticles as mentioned above. Fluorescent values were detected using the TECAN Spark 10 M at the excitation and emission wavelengths of 485/525 nm. GYY4137 was added 1 h before (black squares) or at the (gray circles with black border) same time as the addition of the bioparticles. According to the correlation analysis the presence of GY4137 didn't affect the phagocytosis in the applied concentrations. (C) Opsonized Staphylococcus aureus were incubated with isolated neutrophil granulocytes in the presence or absence of 100 μM hydrogen sulfide and incubated for 20 minutes. The Colony Forming Unit assay was performed and bacterial killing was calculated based on original, extracellular and intracellular bacterium concentrations. Treatment with hydrogen sulfide did not significantly affect bacterial killing compared to nontreated samples

FIG. 5 Histopathological findings in kidney tissues of Rag 2 knockout mice after the passive transfer of splenocytes of MPO immunized MPO deficient mice. (A,B) Black arrows indicate the cellular crescents where proliferating parietal cells, monocytes and other inflammatory cells fill the Bowman capsule. The dotted gray arrow indicates the squashed glomeruli (PAS, X553). (C) Black arrows show the fibrocellular crescents. Figure also shows large cells with distinct large nuclei and the surrounding fibrotic tissue (PAS, X645). (D) As inflammation progresses more fibrotic lesions can be detected in the crescentic area (black arrows). The emergence of fibrotic tissue causes the disappearance of cells that previously appeared in the Bowman's capsule (see 5/A dotted gray arrows). Cells are replaced with fibrin and collagen (PAS, X700). (E,F) In case of persisting serious inflammation the lysis of the Bowman's capsule can be detected (black arrows). In this scenario an array of cells recruited to the site of injury that infiltrate the inflamed area (see 5/A straight white arrows) (PAS, X482). (G,H) Black arrows indicate glomerulosclerosis. This type of injury of the glomeruli cannot be repaired and the function cannot be restored (PAS, X260).

FIG. 6 The effect of GYY4137 sulfide donor treatment in the ANCA mouse model. (A) The number of affected glomeruli were counted in 6 different slices of each kidney sample. Affected glomeruli showed symptoms of cellular, fibrocellular, and fibrotic crescents and also segmental or diffuse sclerotic lesions. Black columns represent the untreated samples and gray dotted columns represent the GYY4137 treated pairs. Each pair received splenocytes from 1 Rag 2 deficient mouse (immunized with BSA or MPO). (B) Columns and numbers indicate the percentage of affected glomeruli in the sulfide donor treated samples compared to their untreated pairs. The gray line represents the 0% baseline (same number of affected glomeruli in treated and untreated mice) and the last column indicates the average percent of all the samples.

FIG. 7 Intraperitoneal treatment of healthy(Rag2−/− immunodeficient) mice with GYY4137 and ATB per os

ATB (60 μmol/kg) was previously tested in ANCA mouse model, administered orally (per os) and proved to be inefficient. In this experiment it has been administered to healthy mice.

GYY4137 (150 μmol/kg) has been administered intraperitoneally into the same breed of animals.

H₂S-level was measured from sera of animals as described in the Examples. Briefly, blood samples were collected from eye corner of mice after isoflurane anesthesia and incubated for 30 minutes at room temperature. The coagulated blood samples were centrifuged, then the sera were collected into fresh tubes. 25 μl of serum samples were mixed with 66 μl of a premixed reagent-buffer solution followed immediately by vigorous vortexing. After exactly 10 min at 20.0° C. the reaction was quenched by TCA and vortexed vigorously. The precipitated proteins were removed and the supernatant was transferred to autosampler vials and cooled. Calibration samples for quantitation were prepared the same way using a standardized NaHS solution and diluted further after the derivatization. HPLC measurement was carried out on C18(2) column using a gradient elution profile using water with 0.1% TFA and acetonitrile with 0.1% TFA. The excitation wavelength of the fluorescence detector was set at 390 nm and the emission wavelength at 475 nm. The preparation was carried out as before with modified chromatography setup [Ditroi, T., et al., Comprehensive analysis of how experimental parameters affect H₂S measurements by the monobromobimane method. Free Radic Biol Med, 2019. 136: p. 146-158.]

Intraperitoneally administered GYY4137 has resulted in a significantly higher serum level than ATB346 administered orally.

FIG. 8 Administration of a donor mix in comparison with GYY4137

A sulfid donor mix (H₂S donor mix) was prepared which comprised GYY4137 (150 μmol/kg, 5 mg/ml strain),N-acetylcysteine (40 μmol/kg, 0.64 mg/ml strain) and pyridoxal 5′-phosphate (20 μmol/kg, 0.49 mg/ml strain).
(A) The H₂S donor mix was administered to healthy mice IP and per os. Serum concentration was measured before sulfide treatment (0 h), and 1 h and 24 h after administration. Surprisingly oral administration resulted in a significantly higher serum level. While serum concentration was the highest at 1 h (after administration), the difference, i.e. ratio of serum levels from oral and IP administration was the highest at 24 h.
(B) GYY4137 at 150 μmol/kg dose, GYY4137 at 750 μmol/kg dose as well as H₂S donor mix comprising GYY4137 at 150 μmol/kg dose, have been administered to healthy mice orally and H₂S serum levels were measured. A higher dose of GYY4137 resulted in a higher serum concentration in comparison with lower dose forms (pure and mix).

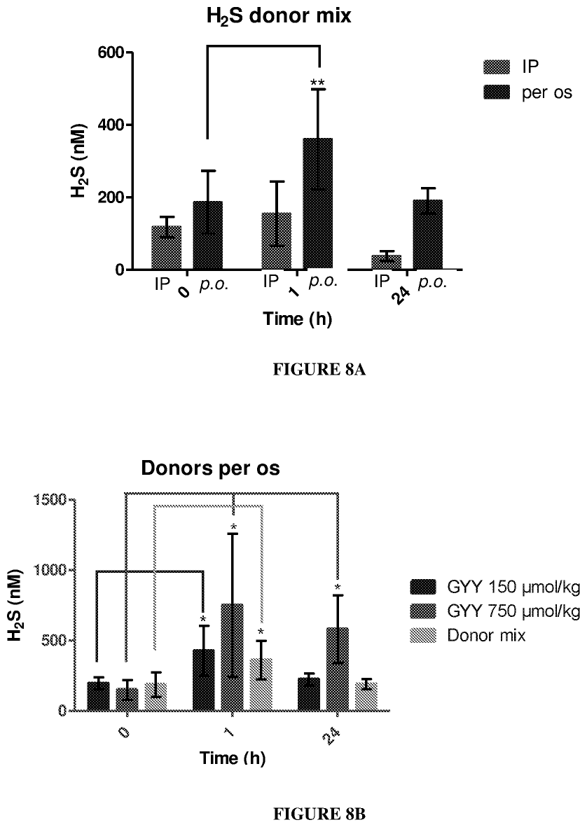

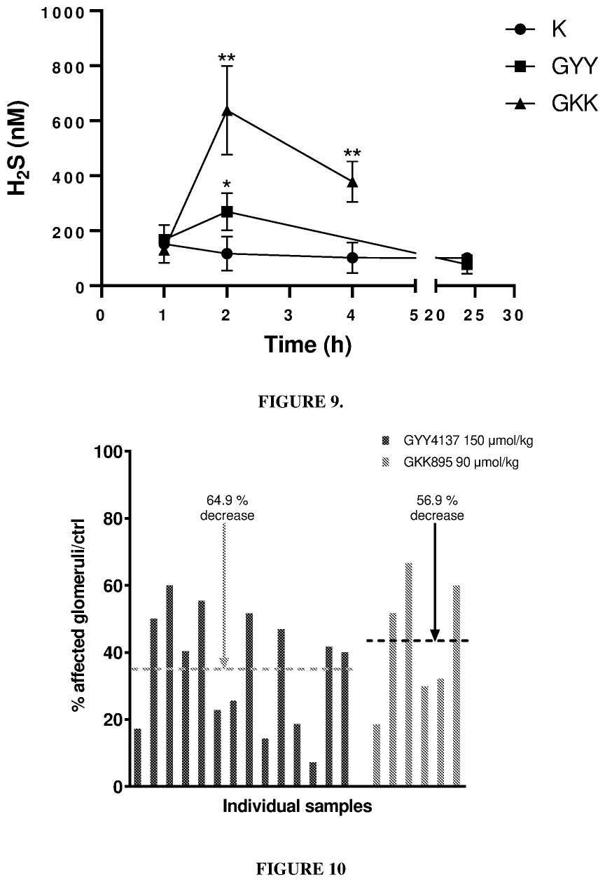

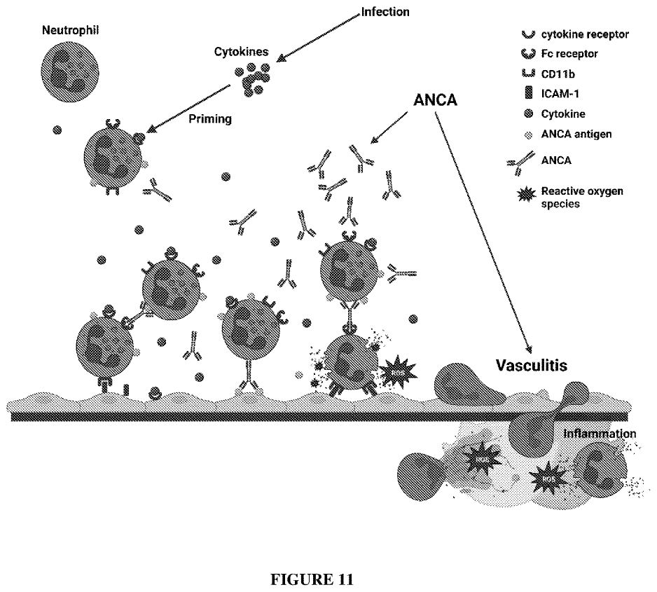

Intraperitoneally administered GYY4137 significantly increased H₂S serum level (see FIG. 7) whereas IP administered H₂S donor mix has not (FIG. 8A).

FIG. 9. Measurement of serum sulfide concentrations of mice treated with GYY4137 or GKK-895. Mice were treated with 150 μmol/kg GYY4137 or 110 μmol/kg GKK-895 intraperitoneally. Blood samples were collected from the eye corner after isoflurane anesthesia at the indicated time points. The concentrations of hydroges sulfide were measured with the monobromobimane assay from the collected blood serum samples. Treatment with both GYY4137 and GKK-895 resulted in significant elevation of sulfide concentrations in the circulation with GKK-895 causing higher measured sulfide values at 2 and 4 hours after injection.

FIG. 10. Comparison of GYY4137 and GKK-895 treatment in mouse kidney samples Kidney samples from the ANCA vasculitis mouse models were prepared for histology analysis and evaluated with light spectroscopy. The percentage of affected glomeruli in treated samples were calculated compared to their control pairs. Samples that were treated with 150 μmol/kg GYY4137 (labeled with dark gray) and also showed decreased renal symptoms were depicted on this figure with an average decrease in affected glomeruli of 56.8%. Samples that were treated with 90 μmol/kg GKK-895 (labeled with light gray) showed an average of 57.6 5% decrease in affected glomeruli compared to control pairs.

FIG. 11 Pathomechanism of ANCA associated vasculitis. (A) Inflammatory cytokines and chemokines (e.g. TNF-alpha) are released due to local or systemic processes which lead to increased expression of endothelial adhesion molecules and neutrophil priming is started. (B) Neutrophil priming results in increased expression of adhesion molecules in neutrophils and induces translocation of ANCA antigens to cell surface. (C) F(ab)2 region of ANCA recognizes ANCA antigens to the cell surface, neutrophils are activated, bind to vascular wall and transmigration is started. (D) ANCA-mediated neutrophil activation induces ROS production and degranulation which leads to vasculitis.

### DETAILED DESCRIPTION OF THE INVENTION

Antineutrophil cytoplasmic autoantibody (ANCA)-associated vasculitides (AAV) have a particularly complex etiological background. The pathogenic role on ANCAs is supported by several studies, including in vitro and in vivo studies as well as clinical trials, and both in vitro and in vivo experiments. Several factors, like neutrophils, complement and effector T cells are also involved in AAV pathogenesis. [Chen, M., Kallenberg, C. ANCA-associated vasculitides-advances in pathogenesis and treatment. Nat Rev Rheumatol 6, 653-664 (2010). https://doi.org/10.1038/nrrheum.2010.158]. A group of patients is of MPO positive serotype and prior art evidence suggest that MPO, as an autoantigen, contributes to the development of MPO positive AAVs.

In a systematic series of experiments the present inventors have studied how sulfide could interfere with these processes and unexpectedly found that H₂S releaser compounds are useful in the treatment of MPO positive serotype AAV.

While neutrophil phagocytosis and bacterial killing was not inhibited in the presence of sulfide, and sulfide and sulfide donor compounds had no effect on PMA induced activation of neutrophil granulocytes, they could efficiently inhibit neutrophil degranulation upon ANCA activation as well as neutrophil activation by isolated IgG from ANCA patients.

The sulfide donors could also inhibit TNF-alpha-induced translocation of ANCA antigen MPO and thereby neutrophil priming wherein active MPO was reported to be a mediator.

Finally, sulfide donor compound protected from kidney damage in a mouse model of MPO-ANCA vasculitis.

The present inventors have also provided evidence that an increased H₂S dose and H₂S level in the serum results in an increased effect showing that the beneficial effect is due to H₂S released. Slow H₂S donors are preferred. Those H₂S donors which, upon administration, provide an increased serum level, can be easily screened by test animals.

Thus, in an unforeseen manner, H₂S donor compounds of the invention act against this autoimmune disease at multiple points whereas normal neutrophil function, so important for a healthy immune system, is not impaired.

The role of hydrogen sulfide is highly controversial in autoimmune inflammatory diseases, and H₂S may easily turn out to be toxic, depending on conditions and concentration. While H₂S was found to be protective against many different processes that contribute to atherogenesis in various models, however, these models seem to have limitations. [Gill Tamás et al. Overview on hydrogen sulfide-mediated suppression ofvascular calcification and hemoglobin/heme-mediated vascular damage in atherosclerosis, Redox Biology, Volume 57, 2022, 102504, ISSN 2213-2317.]

This Janus-faced feature of H₂S places a huge burden on the investigator to arrive at a correct conclusion, whether H₂S is beneficial or not in a specific disease and complex experimentation is required the outcome or result could not be predicted.

Factors and biological processes related to AAV are discussed in more detail below. As a summary see FIG. 11.

Inflammatory cytokines induce the translocation of ANCA antigens, such as MPO and PR3, to the cell surface, which primes the neutrophil cells for antibody-recognition-based activation.

Thus, neutrophil priming includes the translocation of MPO to the cell surface to serve as an antigen for ANCAs, which eventually induces a respiratory burst [Jennette, J. C. and R. J. Falk, Pathogenesis of antineutrophil cytoplasmic autoantibody-mediated disease. Nat Rev Rheumatol, 2014. 10(8): p. 463-73.]. In this pathogenic process ANCA-induced neutrophils infiltrate the walls of small and medium vessels and produce large amounts of cytotoxic reactive oxygen species (ROS) into the extracellular space, in this case into the interstitium of vessel wall. In these processes MPO may be a central player as ANCA-induced release of toxic oxygen radicals and noxious granule enzymes from cytokine-primed neutrophils may mediate vascular inflammation. [Falk, R. J., et al., Anti-neutrophil cytoplasmic autoantibodies induce neutrophils to degranulate and produce oxygen radicals in vitro. Proc Natl Acad Sci USA, 1990. 87(11): p. 4115-9.].

To give some background about neutrophil granulocytes, these cells play an important role in innate immunity and provide a first line protection against pathogenic microorganisms. In an event of infection, they migrate to the site of infection where the killing of pathogens are carried out by phagocytosis and antibacterial proteins. [Jaillon, S., et al. Neutrophils in innate and adaptive immunity. in Seminars in immunopathology. 2013. Springer., Laskay, T., G. van Zandbergen, and W. Solbach, Neutrophil granulocytes as host cells and transport vehicles for intracellular pathogens: apoptosis as infection-promoting factor. Immunobiology, 2008. 213(3-4): p. 183-91..Cassatella, M. A., Neutrophil-derived proteins: selling cytokines by the pound. Advances in immunology, 1999. 73: p. 369-509.] Activated neutrophils produce a repertoire of inflammatory molecules, like CXC and CC chemokines, interleukines, interferons, colony stimulating factors and tumor necrosis factor-α (TNF-α) [Cassatella, M.A., 1999, infra].

The present inventors also investigated the effect of sulfide on the autoimmune inflammatory processes of ANCA vasculitis mediated by neutrophil granulocytes. First it was demonstrated that sulfide inhibits the oxidative burst mechanism induced by ANCA antibodies in a dose dependent manner, regardless of the addition of sulfide before or after priming with TNF-α.

It was known in the art that activation by autoantibodies also induce the release of granular enzymes in the extracellular space [Flint, J., M.D. Morgan, and C.O. Savage, Pathogenesis of ANCA-associated vasculitis. Rheum Dis Clin North Am, 2010. 36(3): p. 463-77.]. Sulfide also interfered with the degranulation upon ANCA activation, indicated by the observation that treatment with the GYY4137 sulfide donor inhibited the release of β-glucuronidase in a dose dependent manner. None of these effects emerged in PMA activated neutrophil samples, suggesting that sulfide acts by inhibiting autoimmune activation of the cells.

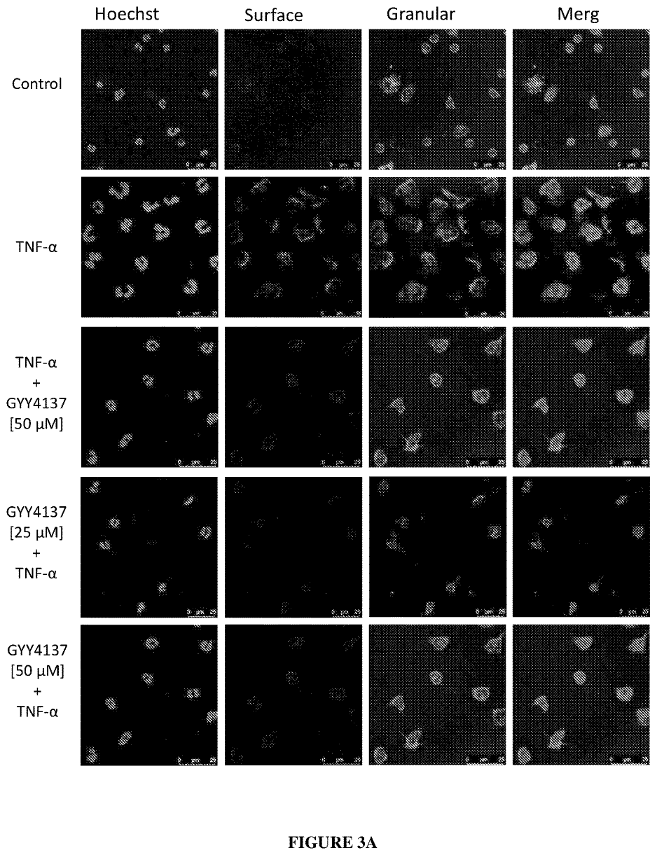

Neutrophil priming by inflammatory cytokines is an important part of this type of activation, therefore the present inventors conducted experiments with TNF-α to investigate the effect of sulfide on this process. The data show that sulfide efficiently inhibited the translocation of MPO to the cell surface, indicating that sulfide may interfere with signaling pathways, that regulate the priming mechanism (FIG. 3).

The present inventors have used the ANCA mouse model published by Xiao and co-workers [Xiao H, Heeringa P, Hu P, Liu Z, Zhao M, Aratani Y, Maeda N, Falk R J, Jennette J C. Antineutrophil cytoplasmic autoantibodies specific for myeloperoxidase cause glomerulonephritis and vasculitis in mice. J Clin Invest. 2002 October; 110(7):955-63] to investigate the potential protective effects of sulfide in MPO-ANCA associated vasculitis. It was shown in the original publication that the most prominent symptoms were manifested in the kidneys. The degree of symptoms of the animals showed high variance, with diverse symptoms and variable number of affected glomeruli. Although, crescent formation and fibrosis in the kidneys were also found in the treated group, quite surprisingly, treatment with GYY4137 lowered the number of affected glomeruli by an average of 64.9%.

While this is believed to be perhaps the best animal model, other models are also known by a person skilled in the art. [Salama A D, Little M A. Animal models of antineutrophil cytoplasm antibody-associated vasculitis. Curr Opin Rheumatol. 2012 January; 24(1):1-7.]

Furthermore, ATB346 and GYY4137 have been tested in ANCA mouse model and in healthy (Rag2 knockout) mice. While IP administered GYY4137, which proved to be active in ANCA-vasculitis mice model and increased H₂S serum level, orally administered ATB346 has neither increased H₂S serum level in healthy mice nor proved to be beneficial in reducing kidney damage in ANCA mouse model under the conditions applied.

Thus, a higher H₂S serum level was in correlation with improving renal condition of model animals.

In a further experiment a sulfide donor mix (H₂S donor mix) comprising GYY4137 preparation (150 gmol/kg), N-acetylcysteine (40 μmol/kg) and pyridoxal 5′-phosphate (20 μmol/kg) were administered to healthy mice IP or per os (FIG. 8/B).

Intraperitoneally administered GYY4137 has significantly increased H₂S serum level (see FIG. 7) whereas IP administered H₂S donor mix has not—a finding was in close correlation with the fact that IP administered GYY4137 improved renal condition in ANCA vasculitis animal model, but the intraperitoneally administered donor mix did not (FIG. 8/A)

The conclusion can be drawn that an increase in H₂S serum level is a condition to actual medical effect in ANCA vasculitis.

Also, the present inventors have surprisingly found that oral administration is preferred over IP administration.

The present data indicate that sulfide donor molecules provide a new treatment option for MPO mediated ANCA vasculitis. The results show that sulfide can reduce oxidative damage in the active phase and may also facilitate induction of remission through inhibition of autoimmune activation of neutrophil granulocytes.

These finding were also corroborated by the experiments with the novel hydrogen sulfide donor GKK-895. Treatment with lower concentration of GKK-895 elevated sulfide concentrations in the blood more than GYY4137 in a higher dose. The elevated blood sulfide levels correlated with advantageous effects in the ANCA mouse model, futher indicating, that there is a strong correlation between elevated blood sulfide levels upon treatment with donor molecules and diminished renal symptoms of ANCA vasculitis.

### Diagnosis of MPO-ANCA Associated Vasculitis Patients

Analysis shows that with sufficient care diagnosis of AAV, despite several subtypes and various symptoms of the disease, is practiced successfully. The 2012 Revised International Chapel Hill Consensus Conference Nomenclature of Vasculitides defined AAV categories microscopic polyangiitis (MPA), granulomatosis with polyangiitis (Wegener's) (GPA), and eosinophilic granulomatosis with polyangiitis (Churg-Strauss) (EGPA) and features of these diseases. [Jennette J C. Overview of the 2012 revised International Chapel Hill Consensus Conference nomenclature of vasculitides. Clin Exp Nephrol. 2013 October; 17(5):603-606.; Jennette J C, et al. 2012 revised International Chapel Hill Consensus Conference Nomenclature of Vasculitides. Arthritis Rheum. 2013 January; 65(1):1-11. doi: 10.1002/art.37715. PMID: 23045170.]

For the present invention detection of MPO positivity and signs of small vessel vasculitis provides a clear definition for diagnostic scope. In a 2016 study by Houben E et al. suggest that a higher ANCA titer and multiple affected organ systems help to discriminate between AAV and other systemic illnesses and that a diagnostic scoring system incorporating these factors should be considered. [Houben E, et al. Diagnosing ANCA-associated vasculitis in ANCA positive patients: A retrospective analysis on the role of clinical symptoms and the ANCA titre. Medicine (Baltimore). 2016 October; 95(40):e5096]

MPO and PR3 ANCA can be, however, positive in a variety of diseases that mimic AAV. A higher ANCA titer and multiple affected organ systems may help to discriminate between AAV and other systemic illnesses in anti-PR3 and anti-MPO positive patients. A diagnostic scoring system incorporating these factors should be considered.

AAV, including GPA or Wegener's granulomatosis, microscopic polyangiitis (MPA), eosinophilic granulomatosis with polyangiitis (EGPA or Churg-Strauss syndrome) as well as other related autoimmune diseases have symptoms in the respiratory tract and kidneys among other organs, are characterized by necrotizing inflammation of small vessels (i.e., arterioles, capillaries, and venules) and the occurrence of pauci-immune (i.e. with little or no deposition of immune complexes in the vessel wall) necrotizing inflammation of the microcirculation, and, in a subset of patients, the presence of antibodies directed against neutrophil and macrophage granule components: namely, myeloperoxidase (MPO-ANCA) and/or proteinase-3 (PR3-ANCA).

[Unizony S. and Stone, J. H. Experimental therapies for vasculitis CHAPTER 42 Oxford Textbook of Vasculitis, Third edition, Oxford University Press 2014 Eds. Ball, Gene V., Fessler, Barri J. and Bridges S. Louis]

The clinical manifestations associated with NCGN include microscopic hematuria with dysmorphic red blood cells and red cell casts, as well as proteinuria.

Patients with systemic vasculitis may present with extra-renal manifestations affecting one or several organ systems, with or without kidney involvement. Commonly involved systems are the upper and lower respiratory tract, skin, eyes, and the nervous system.

[KDIGO 2021 Clinical Practice Guideline for the Management of Glomerular Diseases Kidney International, (2021) 100(45) Supplement, pages S₁ to S₂₇₆]

Systemic symptoms include fever, fatigue, weight loss and muscle or joint aches. Systemic vasculitides are multisystem disorders. For example, the following individual organs may be affected (typical symptoms listed). eyes (e.g. redness, blurred or lost vision), ears (ringing or hearing loss), sinuses (pain, runny or bleeding nose), skin (rashes, ulcers or sores, typically deep and slow to heal), lungs (coughing and chest pain, trouble breathing, dyspnoea or haemoptysis), kidneys (urine comprising protein, foamy urine or blood in urine), nervous system (numbness and/or tingling). Further non-specific symptoms include fever, polymyalgia, polyarthralgia, headache, malaise etc. which overlap with other disease like infections or malignancy. Symptoms may also overlap with those of other inflammatory disease.

Symptoms may manifest in many ways comprising non-specific symptoms and more specific ones. In primary care blood tests may show leukocytosis, thrombocytosis, raised erythrocyte sedimentation rate and C reactive protein values, normochromic-normocytic anaemia, and a raised serum creatinine, the latter is indicative of renal damage. Urinalysis and urinary sedimentation may reveal haematuria and proteinuria. An increased serum creatinine indicates that renal damage has already occurred. Chest radiography may show infiltrates, nodules, or cavitations in the lung parenchyma and is advisable in patients with pulmonary symptoms. [Berden A et al. Diagnosis and management of ANCA associated vasculitis, Clinical Review, BMJ (2012) 344 e26 doi: 10.1136/bmj.e26]

In lack of general reliable serological markers accurate clinical tools may be applied to assess disease activity. For this purpose reliable disease assessment tools have been created. The Birmingham Vasculitis Activity Score (BVAS) and the Vasculitis Damage Index (VDI) have been adopted and used by most research groups involved in clinical trials in vasculitis. These are internationally recognised assessment tools which are also effective to compare clinical trials. The Birmingham Vasculitis Activity Score (BVAS) has also been specifically adapted to Wegener's granulomatosis. [Flossmann O et al. Development of comprehensive disease assessment in systemic vasculitis. Ann Rheum Dis. 2007 μMarch; 66(3):283-92.]

In AAV, a kidney biopsy is also an important option both in case of primary diagnosis and recurrent disease. Biopsy remains the gold standard. Kidney biopsy should always be considered in patients wherein kidney is involved or assumed to be involved as an affected organ.

Nevertheless, in case of a positive MPO- or PR3-ANCA serology and a clinical picture compatible with small-vessel vasculitis with low suspicion for secondary vasculitis, treatment can be started and biopsy may be taken even after the initiation of the treatment.

However, as to relapse of the diseases, ANCA positivity or an increase in ANCA levels are considered only to be modestly predictive of future disease relapse and should not be used to guide treatment decisions.

However, in the present invention the patient group to be treated are MPO-ANCA positive patients. Thus, MPO serotype has to be diagnosed in patients with AAV symptoms.

### Methods Used to Detect MPO and to Determine MPO-ANCA Specificity

An option to treat MPO in patients is indirect immunofluorescence (IIF).

The method of choice for IIF demonstration of ANCA was established in the international consensus statement on testing and reporting of ANCA. [Savige, J. et al. International consensus statement on testing and reporting of antineutrophil cytoplasmic antibodies (ANCA). American Journal of Clinical Pathology, 111, 507-13.]. After preparation the method includes evaluation with an incident light-illuminated fluorescence microscope. The method is available at the European Vasculitis Society (https://vasculitis.org/). [Damoiseaux, J. and Tervaert, J. W. C. Autoantibodies in vasculitis CHAPTER 6 Oxford Textbook of Vasculitis, Third edition, Oxford University Press 2014 Eds. Ball, Gene V., Fessler, Barri J. and Bridges S. Louis]

MPO specificity of ANCA autoantibodies can also be determined by enzyme-linked immunosorbent assays (ELISA). Direct, non-competitive ELISA, wherein MPO is bound to the microtiter plates, is a preferred choice for detection of antigen-specific antibodies. ELISA is a quantitative assay wherein reference standard is to be used. Such standard is available for MPO-ANCA (IUIS-CDC reference preparations) and permit obtaining the results in international units. Several types of direct ELISA are available as known for person skilled in the art. Chemiluminescence type assay is also available. [Damoiseaux, J. and Tervaert, J. W. C. 2014 infra and documents cited therein; see Chapter “Methods used to determine ANCA specificity” ]

Second-generation tests for ANCA detection are so-called capture ELISAs wherein the first coating of the solid phase does not consist of the antigen but of a monoclonal antibody specific for the respective antigen and the antigen preparation is added thereto. It is suggested that this capture of antigens by immobilized antibodies provides a better recognition by the MPO.

Anchor ELISAs, developed later, are the so-called third-generation assays for MPO detection. In these assays purified antigen is coupled to a linker peptide and this linker peptide is used to bind the antigen to the solid phase. Third generation, i.e. anchor ELISA, allowing, similarly to capture ELISA, the maintenance of the three-dimensional structure of the antigen, eventually results in a better sensitivity. [Damoiseaux, J. and Tervaert, J. W. C. 2014 infra and documents cited therein; see Chapter “Methods used to determine ANCA specificity” ].

ANCA test can be purchased commercially, however, their use at present requires trained personnel.

According to the Oxford Textbook of Vasculitis, [Oxford Textbook of Vasculitis Third edition, Oxford University Press 2014 Eds. Ball, Gene V., Fessler, Barri J. and Bridges S. Louis, page 66, Autoantibodies in vasculitis CHAPTER 6, Conclusions] there is international consensus that ANCA should be detected by a combination of IIF and antigen-specific assays, including MPO-ANCA.

A recent 2022 review [Walker Brandon S. et al. Performance of MPO-ANCA and PR3-ANCA immunoassays for the stratification of specific ANCA-associated vasculitis: A systematic review and meta—analysis Autoimmunity Reviews 21 (2022) 103100] conducted a meta-analysis to identify diagnostic accuracy studies using PR3-ANCA or MPO-ANCA for the evaluation of granulomatosis with polyangiitis (GPA), microscopic polyangiitis (MPA), and eosinophilic granulomatosis with polyangiitis (EGPA).

The present invention is to be used in the MPO serotype of AAV patients, therefore MPO-ANCA positive patients are to be treated. In a preferred variant therefore MPO-ANCA is detected in the AAV patient in the present invention. In a particular embodiment MPO-ANCA is detected in the subject and said subject is treated with a compound for use according to the invention.

In a preferred embodiment the AAV clinical subtype is GPA, MPA, or EGPA.

In a preferred embodiment the IIF and an antigen-specific assay, e.g. ELISA is performed to detect MPO-ANCA. In particular, ELISA is a first, second or third generation ELISA as explained above.

In an embodiment disease progress is monitored in said AAV patient of MPO-ANCA serotype. In this regard an elevation of MPO level, measured in two different time-point, is indicative of a relapse and treatment is to be adjusted. In this regard, in a preferred embodiment, IIF in combination with ELISA, is performed or ELISA, preferably a second or third generation ELISA is performed.

### Compounds Useful in the Invention

### GYY4137

An example of sulfide donor compounds used by the present inventors is GYY4137 (morpholin-4-ium 4 methoxyphenyl(morpholino) phosphinodithioate). It is often used in its dichloromethane complex (Formula 1). [Rose, P. et al. GYY4137, a Novel Water-Soluble, H₂S-Releasing Molecule, Methods in Enzymology, Volume 554, 2015 Elsevier Inc ISSN 0076-6879].

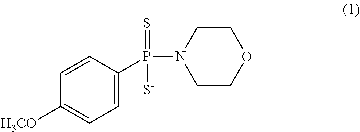 [SMILES: COc1ccc(P(=S)([S-])N2CCOCC2)cc1]

Chemical synthesis of GYY4137 and release of H₂S in vitro and in vivo is described by Li L et al. [Li L, et al. Characterization of a novel, water-soluble hydrogen sulfide-releasing molecule (GYY4137): new insights into the biology of hydrogen sulfide. Circulation. 2008 May 6; 117(18):2351-60.]

The authors have also described that GYY4137 released H₂S slowly both in aqueous solution in vitro and after intravenous or intraperitoneal administration in anesthetized rats in vivo.

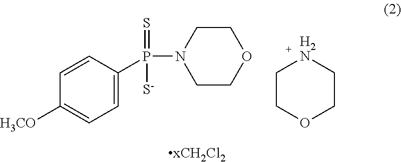

Typically, GYY4137 was used in the art by intraperitoneal administration.

The present inventors have prepared both intraperitoneal (ip) and oral (per os) of formulations GYY, tested in mice (see the Examples) and unexpectedly found that oral formulations resulted in higher serum levels. Plausibly, oral formulation is more preferred and effective for the treatment of MPO-ANCA vasculitis than intraperitoneal applications.

Derivatives of GYY4137 may also be useful in the present invention.

Such derivatives are described e.g. by Huang et al. [Huang, C. W. et al., A novel slow-releasing hydrogen sulfide donor, FW1256, exerts anti-inflammatory effects in mouse macrophages and in vivo., Pharmacological Research 113 (2016) 533-546].

Examples for such compounds are dithiophosphor diamines, which are fast H₂S releasers and benzo[d][1,3,2]oxazaphospholes which are medium and/or slow releasers (slow-medium releasers).

A further option are derivatives of GYY4137 which are slow releasers and which

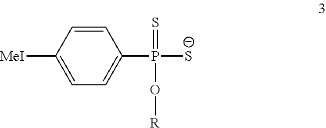

### Aspirin Derivatives

Anethole trithione (ADT-OH, formula 4) is a known H₂S releasing compound.

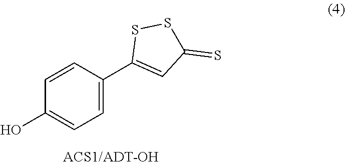 [SMILES: Oc1ccc(-c2cc(=S)ss2)cc1]

While anethole trithione is a H₂S donor itself, it is also useful because it can be attached to other compounds e.g. non-steroidal anti-inflammatory drugs (NSAIDs) such as aspirin or other NSAIDs. [Song et al., Hydrogen sulfide donors in research and drug development, Issue Med. Chem. Commun., 2014, 5, 557; IDS]. From these compounds ADT-OH is released in vivo which in turn works as a H₂S donor whereas the other counterpart of the molecule, here an aspirine derivative, has its own effect as well.

However, several other type of NSAIDs, like nonselective cyclooxygenase (COX-1 and COX-2) inhibitors such as diclofenac and naproxen, may have long term harmful gastrointestinal or renal effect on renal conditions and therefore are less advantageous in the present invention (wherein AAV disease itself have anti-renal effect itself). [Li, L. et al. Anti-inflammatory and gastrointestinal effects of a novel diclofenac derivative. Free Radicals Biol. Med., 2007, 42, 706.]

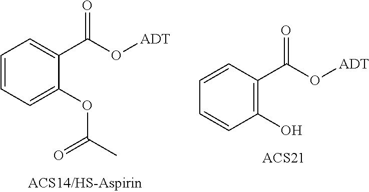

ADT-OH, ACS14 and ACS21 can be prepared and administered orally as described by Sparatore Anna, et al. [Sparatore A, et al. Pharmacological profile of a novel H₂S-releasing aspirin Free Radical Biology & Medicine 46 (2009) 586-592].

Formulation of the compound were prepared by using 2 ml/kg of a mixture containing 0.5% w/v carboxymethylcellulose and DMSO, 9/1, v/v, and the active agent, which were provided in a dose as follows: ACS14 (50 mg/kg), aspirin (23 mg/kg), ACS21 (45 mg/kg), salicylic acid (18 mg/kg), and ADTOH (29 mg/kg). Administration was once a day for 7 consecutive days. The doses of ACS14, ACS21, salicylic acid, and ADTOH used in these experiments are equimolar to that of aspirin. [Sparatore, A. et al, 2009, infra].

### Examples of Natural H₂S Donors

Natural H₂S donors may also be applied in the present invention.

Example for such compounds are given below which can be purchased from various exemplary providers.

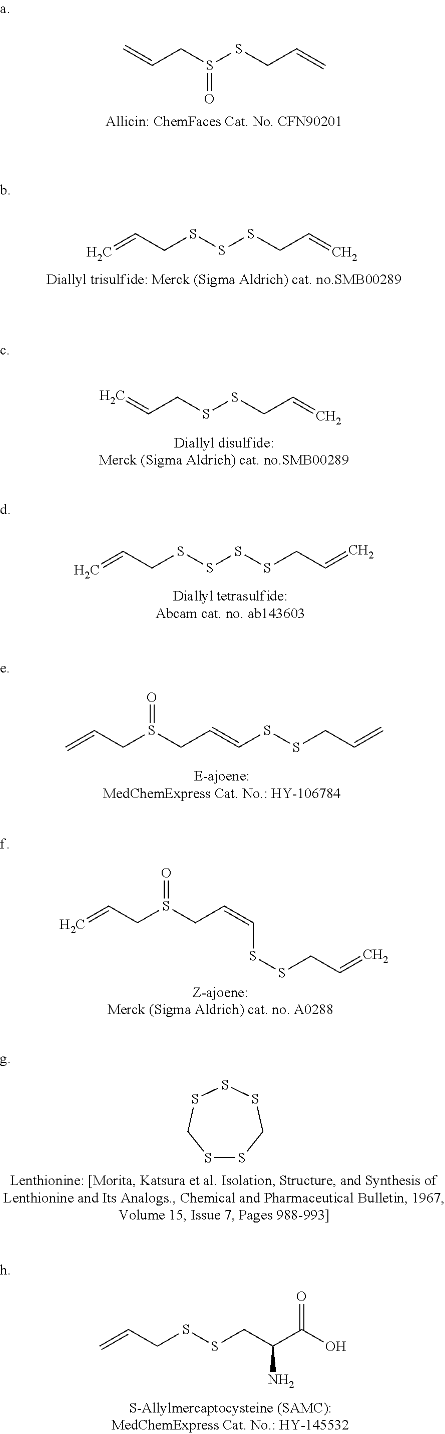

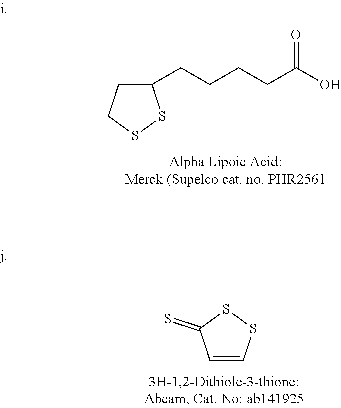 [SMILES: O=C(O)CCCCC1CCSS1 ; S=c1ccss1]

Further exemplary H₂S-donors which do not comprise an —S—S— group are e.g. the following:

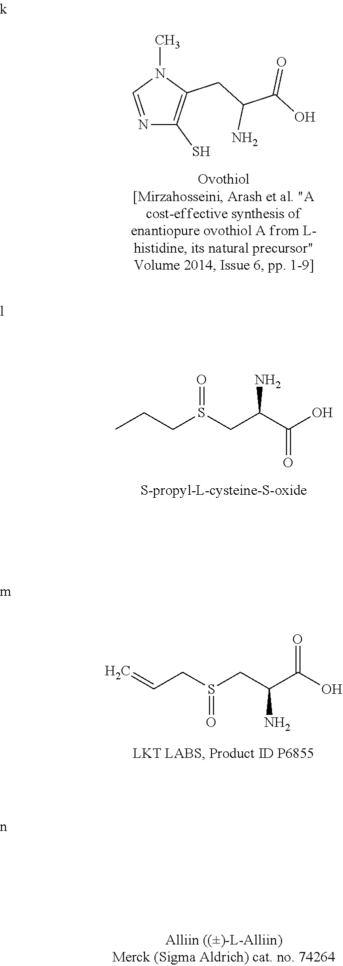

### Pharmaceutical Preparations of the Invention and Administration

Preparation of compositions or pharmaceutical preparation from the present compounds for use is within the skills of a person skilled in the art.

For long time storage it is advantageous if the compositions are stored in dry form, e.g. crystallized or lyophilized.

Appropriate vehicles are known in the art. A typical vehicle used is e.g. carboxymethyl-cellulose.

Furthermore, several compounds may be sensitive to hydrolysis which advises towards using a buffer in particular in injectable formulations, however, even in dry formulations avoiding formation of acidic or basic pH may be advisable.

Injectable formulation can be reconstituted before use and therefore only soluble components are to be used.

Poor solubility resulting in limited drug loading can occur as a problem which has to be solved.

Typical excipients may include the following categories and examples: Disintegrants, like crosslinked polymers, polyvinylpyrrolidone (crospovidone), crosslinked sodium carboxymethyl cellulose (croscarmellose sodium), in particular the latter.

Binders, including for example
saccharides e.g. disaccharides (lactose, saccharose); polysaccharides (e.g. cellulose, starches etc.), modified polysaccharides such as microcrystalline cellulose, cellulose ethers, or derivatized saccharides, e.g. carboxymethyl cellulose etc. may be useful;
sugar alcohols such as xylitol. sorbitol or mannitol; in particular mannitol (E421)
protein type binder, like gelatin; (in particular in light gelatin capsule).

Lubricants, like magnesium stearate, or other stearate derivative (or talc or silica etc.) can be applied.

Polymers having the role of a stabilizer, surfactant thickening agent, solubility enhancer, e.g. Povidone (polyvinylpyrrolidone, PVP) or other synthetic polymers, like polyethylene glycol (PEG), preferably Povidone.

In the present invention the compounds used are typically rather hydrophobic, light sensitive compounds. Thus, when formulating them into pharmaceutical compositions these problems should be considered. It is advantageous if the compositions are protected from light.

A preferred formulation is capsule, e.g. light gelatin capsule or hard capsule wherein the active agent is protected from light. In an embodiment the package should protect the azidated compounds from light. Non-transparent capsules are preferred for oral administration

An example is Lonza Capsugel of TiO₂-free light-protected capsules (See [Lonza Press Release “Lonza Expands its Capsugel® Capsule Offering to Include Titanium Dioxide-Free White Hard Gelatin Capsules” May 9, 2022, Basel, Switzerland]).

Methods for encapsulation or incorporation into polymeric matrices, including nano- and micro-particles, with increased loading are also known and related compound is disclosed as a nanoemulsifed formulation and the like are known in the art.

Below the invention is further characterized by way of non-limiting examples.

### Materials and Methods

### Preparation of Sodium Sulfide and GYY4137 Sulfide Donor Stock Solutions

Sodium sulfide stock solutions were prepared freshly, sterile filtered (pore size 0.2 μm) and stored on ice in covered plastic tubes until use. A relatively large crystal of sodium sulfide was rinsed then dissolved in ultrapure degassed water. The stock solution was diluted appropriately and the concentration was measured at 240 nm. Contamination by sulfide oxidation products was checked by adding 400 μM DTNB and measuring the absorbance at 560 nm. The stock solution was appropriate for use when the difference between the calculated concentrations from the two measured absorbances was less than 5%. GYY4137 (SigmaAldrich, product no. SML0100, PubChem Substance ID:329825158) was dissolved in DMSO or DTNB in an appropriate concentration and sterile filtered with a pore size of 0.2 μm [Palinkas, Z., et al., Interactions of hydrogen sulfide with myeloperoxidase. Br J Pharmacol, 2015. 172(6): p. 1516-32.].

GYY4137 (P-(4-Methoxyphenyl)-P-4-morpholinyl-phosphinodithioic acid, compound with morpholine (1:1), morpholin-4-ium 4-methoxyphenyl(morpholino) phosphinodithioate), e.g. in the form of dichloromethane complex, can be purchased, for experimental purposes, e.g. from Merck (Sigma-Aldrich), Cat. No. SML0100.

### Human Samples

All human samples were prepared from peripheral venous blood obtained with informed consent from healthy donors or ANCA patients with active disease. Handling samples were carried out according to the national regulations [Ethical decision: BPR-021/00084-2/2014; Research No.: 84/2014, Project No.: 4678-2016 1,21.

Isolation of Immunoglobulins from Human Venous Blood

Immunoglobulins were isolated using Protein G Agarose resin according to the protocol provided by the manufacturer. Blood serum samples were diluted with binding buffer (1:1), loaded onto the agarose column, mixed and incubated for 2 hours on room temperature. The column was washed with 15 ml binding buffer then IgG fraction was recovered with acidic elution using 100 mM glycine solution (pH 2.5). 0.5 ml flowthrough fractions were collected and the pH was neutralized with 1 μM phosphate buffer (pH 7.5). Fractions with the highest protein concentrations were pooled and dialyzed against 2×500 ml HBSS without Ca²⁺ and Mg²⁺. Protein concentrations were measured using Bradford reagent and the solution was sterile filtered (pore size 0.2 μm).

### Isolation and Activation of Neutrophil Granulocytes

Peripheral venous blood was obtained from healthy adult donors with informed consent. Red blood cells were sedimented on 1% dextran solution (1% dextran dissolved in DPBS), the pale yellow supernatant was pipetted to a plastic tube and centrifuged for 5 minutes at 500 g. The supernatant was discarded, then cells were resuspended in 10 ml DPBS and centrifuged at 500 g for 5 minutes. Polymorphonuclear cells were resuspended in 5 ml DPBS and layered over 5 ml Hystopaque 1077 solution and centrifuged for 30 minutes at 800 g without break. After gradient centrifugation with Hystopaque the polymorphonuclear cells were settled at the bottom of the tube together with the remaining red blood cells. Red blood cells were lysed with 0.2% NaCl solution, then isosmotic conditions were restored by adding equal volume of 1.6% NaCl solution. The cells were washed with DPBS, resuspended in HBSS solution and put on ice until use. The viability and cell concentration was measured using trypan blue solution [Boyum, A., Isolation of lymphocytes, granulocytes and macrophages. Scand J Immunol, 1976. Suppl 5: p. 9-15.] The polymorphonuclear cell suspension was used under 2 hours after preparation. Neutrophil granulocytes were activated with 100 ng/ml 12-phorbol 13-myristate acetate (PMA) and incubated for 20 minutes in the appropriate assay buffer. Equal volume (1 μl) of DMSO was added to all of the control samples. For the autoimmune activation of cells 300 gg/ml isolated IgG from ANCA patients or healthy donors were used. First, cells were pre-treated with different concentrations of GYY4137 for 5 minutes, then 2 ng/ml TNF-α was added to the samples and incubated for 15 minutes. After priming with TNF-α, samples were treated with purified IgG and incubated for 60 minutes unless otherwise specified.

### Effect of Hydrogen Sulfide on the NOX2 Activity of Neutrophil Granulocytes

The Fox reagent was prepared (100 μM xilenol orange, 250 μM ammonium iron(II) sulfate, 100 mM sorbitol and 25 mM sulfuric acid diluted in 30 ml ion exchanged water) according to Wolff's [Jiang, Z. Y., et al. Lipid hydroperoxide measurement by oxidation of Fe2+ in the presence of xylenol orange. Comparison with the TBA assay and an iodometric method. Lipids, 1991. 26(10): p. 853-856.] published protocol, and stored on ice until use. Neutrophil granulocyte samples were prepared in the L-tyrosine assay buffer as discussed earlier using PMA as the activating agent and adding 1 mM Na-azide to inhibit MPO activity and prevent degradation of hydrogen peroxide. Supernatants were diluted by 50× with 50 mM phosphate buffer, 50 μl of FOX assay reagent was added to 140 μl of diluted sample and mixed thoroughly. For standard curve 140 μl hydrogen peroxide dilution series (of 0.5-4 μM) in DPBS was mixed with 50 μl FOX reagent. Samples and standards were incubated in room temperature for 40 minutes and the optical density was measured at the wavelength of 560 nm.

### Measurement of the Effect of Sulfide on Neutrophil Granulocyte Activation

The neutrophil oxidative burst was measured based on the activity of NOX2 upon activation with PMA or IgG from healthy donors or MPO-ANCA patients. WST-1 reagent was used that reacts with the superoxide produced to the extracellular space [Ngamwongsatit, P., et al., WST-1-based cell cytotoxicity assay as a substitute for MTT-based assay for rapid detection of toxigenic Bacillus species using CHO cell line. Journal of Microbiological Methods, 2008. 73(3): p. 211-215.; Tan, A. S. and M. V. Berridge, Superoxide produced by activated neutrophils efficiently reduces the tetrazolium salt, WST-I to produce a soluble formazan: a simple colorimetric assay for measuring respiratory burst activation and for screening anti-inflammatory agents. J Immunol Methods, 2000. 238(1-2): p. 59-68.]. The activation of neutrophils was carried out in the presence of 300 μM WST-1 and 20 μg/ml catalase and in the presence or absence of different concentrations of sulfide (5-50 μM) or GYY4137 sulfide donor (20-100 μM). Cells were either incubated in the presence of sulfide before or after the addition of TNF-α for 5 minutes. PMA activated samples were incubated for 20 minutes, ANCA activated samples for 90 minutes and all samples were put on ice for 5 minutes to slow down reactions. All samples were centrifuged at 2000 g for 5 minutes then supernatants were pipetted into a 96-well plate and absorbances were measured at 450 nm. The concentrations of produced superoxide were calculated using the molar extinction coefficient of WST-1 (37×10³ μM⁻¹ cm⁻¹) considering that 2 mol superoxide is needed for the reduction of 1 mol WST-1.

### Effect of Sulfide on Neutrophil Priming by TNF-α

24-well sterile plate including glass sterile coverslips was treated with 0.1% gelatin at 37° C. for 1 hour. The plate was washed with 500 μl DPBS 3 times, 2.5×10⁵ neutrophils were incubated for 30 minutes, then treated with appropriate concentrations of GYY4137 for 5 minutes prior or at the same time as the addition of 10 ng/ml TNF-α. Neutrophils were primed for 15 minutes in the presence or absence of GYY4137 at 37° C. The supernatants were discarded and the cells were fixed with 3.7% paraformadehyde for 15 minutes at room temperature, then nonspecific antigens were blocked with 5% goat serum overnight at 4° C. Cells were washed with 500 μl DPBS 3 times then the surface MPO was labeled with rabbit anti-MPO antibody (1:2000) at room temperature for 1 hour. The wells were washed 3 times, then labeled with goat anti-rabbit Alexa Flour 488 (1:500) secondary antibody at room temperature in the dark for 1 hour. Cells were then permeabilized with 0.1% Triton X for 15 minutes in the dark in order to label the intracellular MPO. Cells were then washed 3 times then labeled with rabbit anti-MPO antibody as mentioned above. After the washing step intracellular MPO was labeled with goat anti-rabbit Alexa Fluor 568 (1:500) secondary antibody for 1 hour in the dark. For nucleus staining 0.5 ng/ml Hoechst 33258 were used for 15 minutes at room temperature. Cells were washed then the coverslips were mounted with Prolong™ Glass Antifade Mountant on glass slides and incubated for 15 minutes. Microscopic analysis was carried out using STED microscopy system.

### Measurement of Neutrophil Degranulation in the Presence of Sulfide or Sulfide Donor

Neutrophil granulocytes in the final concentration of 2×10⁶ cells/ml were incubated with 5 μM cytochalasin B in HBSS for 10 minutes in 37° C. Different concentrations of sulfide or GYY4137 was added to the cells and incubated for 5 minutes. In case of PMA activation sodium sulfide (0-50 μM) was used and after the 5 minutes the cells were activated with PMA. For the autoimmune activation with IgG the neutrophils were treated with GYY4137 sulfide donor (0-80 μM) and samples were subsequently primed with 4 ng/ml TNF-α for 5 minutes then incubated with ANCA or healthy IgG (300 μg/ml) for 30 minutes. For positive control 1% Triton X were added to the samples, incubated for 20 minutes then vortexed. All samples were put on ice for 5 min, centrifuged at 2000 g for 5 minutes then 100 μl of supernatants were pipetted into a 96-well plate with 100 μl of 1 mM phenolphthalein-glucuronide dissolved in 100 mM Na-acetate solution and incubated for 19 hours. The enzymatic reaction was stopped using 100 μl of 400 mM glycine buffer with 200 mM NaCl creating a pink colour of phenolphthalein that was measured by spectrophotometry at 540 nm. One unit of enzyme activity was the amount which liberated 10 μg phenolphthalein in 19 hours. For standard curve 1 mg phenolphthalein was dissolved in 200 μl 96% drum grate ethanol, vortexed then 800 μl ion exchanged water was added to make 1 mg/ml. Dilute and use as soon as possible. Abb 100 μl dilutions of phenolphthalein, 100 μl HBSS and 100 μl glycine buffer. Protein levels of neutrophil supernatants of PMA activated samples were analyzed with SDS-PAGE and silver staining.

### Effect of Sulfide on Neutrophil Phagocytosis

Phagocytosis experiments were carried out with the Vybrant™ Phagocytosis Assay Kit based on manufacturers instruction. Briefly, Fluorescent-labeled Escherichia coli K-12 BioParticles, and trypan blue stock solutions were prepared. Neutrophils at 4*10⁴ cells/well were incubated in the presence or absence of GYY4137 (20-100 μM) for 1 h in black 96-well plates to allow the cells to adhere. After incubation time the supernatants were removed and 100 μl of prepared fluorescent BioParticles suspension was added and incubated for 2 hours at 37° C. in the presence or absence of GYY4137 (20-100 μM). In the two different experimental conditions cells were treated with GYY4137 1 hour or 5 minutes prior to the addition of the fluorescent BioParticles suspension. After 2 hours the BioParticle solution was removed, 100 μl trypan blue solution was added immediately, and incubated for 1 minute at room temperature. Trypan blue was removed and 100 μl HBSS was added to each well. Fluorescence was measured at the excitation and emission wavelengths of 480 and 520 nm respectively. Samples for microscopy analysis were prepared similarly with the cell number of 2×10⁵/well in EZ slide chamber slides.

### Effect of Sulfide on Neutrophil Bacterial Killing

Staphylococcus aureus was cultured on columbia sheep blood agar plate, incubated overnight at 37° C., and then stored at 4° C. For experiments, a single colony was transferred from the plate into 15 ml of nutrient broth and incubated overnight at 37° C. and then centrifuges at 1000 g for 5 minutes and washed twice in PBS.

The concentrateion was measured using a curve of turbidity (A550 of 0.2 approximately 1×10⁸/ml). For opsonization, bacteria were suspended at 1×10⁷/ml in PBS with 10% human blood serum (pooled from at least 5 healthy donors). The tubes were rotated end-over-end at 6 rpm for 20 mm at 37° C.

The effect of Hydrogen sulfide on bacterial killing was measured using a modified form of the Colony Forming Unit (CFU) assay [Hampton M B, et al. A single assay for measuring the rates of phagocytosis and bacterial killing by neutrophils. J Leukoc Biol. 1994 February; 55(2):147-52. doi: 10.1002/jlb.55.2.147. Erratum: Hampton M B, et al. J Leukoc Biol 1994 July; 56(1):104. PMID: 8301210.]. For experimental reactions 500 μL of isolated human neutrophils (1×107/mL prewarmed to 37° C. for 10 min) were prepared with or without 100 μM NaHS and added to 500 μL freshly opsonised bacteria (1×108/mL) and 50 μL prewarmed serum (10%). For the control reaction, neutrophils were replaced with 500 μL Hank's buffer. The reactions were incubated at 37° C. with end-over-end rotation (6 rpm). 50 μL samples were pipetted after 20 minute incubation into 950 μL ice-cold PBS to stop neutrophil activity. Samples were centrifuged at 100 g for 5 minutes, 4° C. (to sediment neutrophils but not bacteria) and the neutrophil pellets were washed twice more with 1 mL ice-cold PBS. Supernatants were collected in each step to measure bacteria that were not taken up by neutrophils. The pellets were resuspended in 2.5 mL water brought to pH 11.00 with NaOH for 5 minutes then vortexed thoroughly. Then each sample including controls, supernatants and intracellular bacteria were diluted in in water (pH 11) to give approximately 50 colonies per half plate when plated onto Colombia Sheep Blood Agar and the plated were incubated overnight at 37° C. Colonies were counted and the number of intracellular colonies were corrected with the control and supernatant samples to compare killing in untreated and hydrogen sulfide treated samples.

### Mice

### ANCA Vasculitis Animal Model

Breeding pairs of Black 6 recombinase-activating gene-2-deficient (Rag2−/−) mice and MPO-deficient (Mpo−/− mice) were purchased from Jackson Laboratories and maintained by the Department of Experimental Pharmacology of the National Institute of Oncology (ethical decision: PE/EA/00419-4/2022). Mice that were 10-14 weeks were used in all experiments and different groups were used as presented in the table below (Table 1).

| | Control | anti-BSA | anti-MPO |
|---|---|---|---|
| | Splenocytes | splenocytes | splenocytes |
| PBS | 2 | 4 | 10 |
| 150 μmol GYY4137/kg | 2 | 4 | 10 |
| 90 μmol/kg GKK-895 | 2 | — | 6 |

Animal experiments were carried out according to Falk's published protocol [Xiao, H., et al., Antineutrophil cytoplasmic autoantibodies specific for myeloperoxidase cause glomerulonephritis and vasculitis in mice. J Clin Invest, 2002. 110(7): p. 955-63]. MPO knockout mice were immunized with mouse MPO based on the following protocol. On day 1 200 μl of 20 μg murine MPO (mMPO) or BSA in total with Complete Freud's Adjuvant were injected to MPO KO mice. 30-40 μl were injected to the backfoot footpads and 120-160 μl were injected subcutaneously in the abdominal region. On day 14 20 μg mMPO/BSA were injected IP with incomplete Freud's Adjuvant and on day 28 mice were boosted with 20 μg mMPO/BSA in DPBS buffer IP. 10 days after boost blood was drawn from eye corner under isoflurane anesthesia and antibody titers were checked by anti-MPO ELISA. If titer were low mice were boosted again with 20 μg mMPO or BSA and titer were checked again 7 days later. If titers were appropriate mice were terminated and splenocytes were isolated. Spleen was removed, gently homogenized in RPMI 1640 filtered with 70 μM sterile filter and washed twice with cold RPMI 1640. The remaining red blood cells were lysed with Red Blood Cell Lysis Buffer according to manufacturer's instructions. Cells were resuspended in RPMI 1640 in 600 μl. Cells were counted using trypan blue solution, the total cell count varied between 3×10⁷ and 6×10⁷. Splenocytes derived from 1 mouse were injected into 2 Rag2−/− immunodeficient mice intravenously. The pair were treated with 100 μg/kg GYY4137 or DPBS every other day for 5 weeks. After 4 weeks antibody titers were checked with anti-MPO ELISA. After 5 weeks mice were euthanized with isoflurane, lung and kidney tissue samples were fixed in 10% paraformaldehyde and prepared for light microscopy analysis. Lung samples were stained with hematoxylin eosin (H&E), kidney samples were stained with H&E, periodic acid Schiff stain and selected samples were stained with Masson's trichrome.

In an alternative experiment the pair were treated with 90 μmol/kg GKK895 or DPBS 3 times a week for 3 weeks. GKK895 is a derivative of GYY4137. After 3 weeks mice were euthanized with isoflurane, kidney tissue samples were fixed in 10% paraformaldehyde and prepared for light microscopy analysis. Kidney samples were stained with H&E and periodic acid Schiff stain.

To compare GYY4137 and GKK-895 treatment in mouse kidney, kidney samples from the ANCA vasculitis mouse models were prepared for histology analysis and evaluated with light spectroscopy. The percentage of affected glomeruli in treated samples were calculated compared to their control pairs. Samples that were treated with 150 μmol/kg GYY4137 (labeled with dark gray) and also showed decreased renal symptoms were depicted on this figure with an average decrease in affected glomeruli of 64.9%. Samples that were treated with 90 μmol/kg GKK-895 (labeled with light gray) showed an average of 56.9% decrease in affected glomeruli compared to control pairs (FIG. 10). The figure only shows GYY treated samples were the treatment was effective.

### Healthy (Rag2−/− Immunodeficient) Mice and H₂S Serum Level Measurement

Healthy mice Rag2−/− immunodeficient mice, see above, were used for IP and per os administration experiments and held and treated as described above, mutatis mutandis.

Blood samples were collected from eye corner of mice under isoflurane anesthesia and incubated for 30 minutes at room temperature. The coagulated blood samples were centrifuged at 2000 g for 10 minutes then the sera were collected into fresh tubes. 25 μl of serum samples were mixed with 66 μl of a premixed reagent-buffer solution (65 μl 200 mM HEPES pH 8.2+1 μl 100 mM MBB in ACN) followed immediately by vigorous vortexing. After exactly 10 min at 20.0° C. the reaction was quenched by the addition of 5 W 50% TCA (w/v) and vortexed vigorously. The precipitated proteins were removed by centrifugation at 3000 g for 5 min and the supernatant was transferred to autosampler vials and held at 4° C. Calibration samples for quantitation were prepared the same way using a standardized NaHS solution and diluted further after the derivatization. For the HPLC measurement 3 μl was injected on a Phenomenex Luna C18(2) 250×2 mm 3 μm column using a gradient elution profile using water with 0.1% TFA and acetonitrile with 0.1% TFA.The excitation wavelength of the fluorescence detector was set at 390 nm and the emission wavelength at 475 nm. The preparation was carried out as before with modified chromatography setup [Ditroi, T., et al., Comprehensive analysis of how experimental parameters affect H₂S measurements by the monobromobimane method. Free Radic Biol Med, 2019. 136: p. 146-158.].

Measurement of Serum Sulfide Concentrations of Mice Treated with GYY4137 or GKK-895

Measurement of serum sulfide concentrations of mice treated with GYY4137 or GKK-895 μMice were treated with 150 μmol/kg GYY4137 or 110 μmol/kg GKK-895 intraperitoneally. Blood samples were collected from the eye corner under isoflurane anesthesia at the indicated time points. All blood samples were incubated at room temperature for 30 minutes then centrifuged at 2000 g for 10 minutes. The concentrations of hydrogen sulfide were measured with the monobromobimane assay from the collected blood serum samples [Ditroi, T., et al., Comprehensive analysis of how experimental parameters affect H2S measurements by the monobromobimane method. Free Radic Biol Med, 2019. 136: p. 146-158.]. Treatment with both GYY4137 and GKK-895 resulted in significant elevation of sulfide concentrations in the circulation with GKK-895 causing higher measured sulfide values at 2 and 4 hours after injection.

It can be concluded that serum level of H₂S is an important factor in respect of the effect of these compounds.

### The Effect of Sulfide on the Production of Reactive Oxyglen Species by 12-Phorbol 13-Myristate Actetate (PMA)-Activated Neutrophils

First we have investigated the effect of sulfide on the activation of neutrophils by 12-phorbol 13-myristate acetate PMA.

Fox reagent was prepared (100 μM xylenol orange, 250 μM ammonium iron(II) sulfate, 100 mM sorbitol and 25 mM sulfuric acid diluted in 30 ml ion exchanged water) and stored on ice until use. Neutrophil granulocyte samples were prepared in the in Hank's Balanced Salt Solution (without phenol red) using PMA as the activating agent and adding 1 mM sodium azide to inhibit MPO activity and prevent degradation of hydrogen peroxide. Supernatants were diluted by 50× with 50 mM phosphate buffer, 50 μl of FOX assay reagent was added to 140 μl of diluted sample and mixed thoroughly. For standard curve 140 μl hydrogen peroxide dilution series (of 0.5-4 μM) in DPBS was mixed with 50 μl FOX reagent. Samples and standards were incubated in room temperature for 40 minutes and the optical density was measured at the wavelength of 560 nm,(see Methods and [Wolff, S. P., Ferrous Ion Oxidation in Presence of Ferric Ion Indicator Xylenol Orange for Measurement of Hydroperoxides. Oxygen Radicals in Biological Systems, Pt C, 1994. 233: p. 182-189]). H₂O₂ production, measured by the FOX assay by PMA-activated neutrophils, was not affected in the applied sulfide (5-50 μM) concentration range (FIG. 1/A).

### Effect of Sulfide on the Oxidative Burst of PMA or ANCA Activated Neutrophils

In case of MPO-ANCA activation of neutrophils, the Fox assay could not be used to evaluate peroxide formation because it involves the addition of sodium-azide, which could interfere with the antigen function of cell surface MPO, therefore we applied a different method. The production of superoxide was measured using the WST-1 reagent [Tan, A. S. and M. V. Berridge, Superoxide produced by activated neutrophils efficiently reduces the tetrazolium salt, WST-1 to produce a soluble formazan: a simple colorimetric assay for measuring respiratory burst activation and for screening anti-inflammatory agents. J Immunol Methods, 2000. 238(1-2): p. 59-68]. The reduction of WST-1 to the reporter yellow product was not inhibited by sodium-sulfide (FIG. 1/B) or the sulfide donor GYY4137(FIG. 1/C) in PMA-stimulated neutrophil samples. Cells that were treated with isolated IgG from ANCA patients produced significantly higher concentrations of superoxide than samples treated with IgG isolated from healthy donors (p=<0.05).

Surprisingly, the sulfide donor GYY4137 inhibited superoxide production in a dose dependent manner with the IC₅₀ value of 46.7 μM (FIG. 1/D). This effect was more pronounced, when GYY4137 was added 5 minutes before priming with TNF-α (FIG. 1/D, black colunms) compared to samples that were treated with the sulfide donor after priming with slightly a higher IC₅₀ value of 62.6 μM (FIG. 1/D, patterned colunms). These results indicated, that sulfide may interfere with the neutrophil priming process too. These observations corroborate that sulfide has no effect on NOX2 activity, considering that treatment with sodium sulfide or GYY4137 had no effect on PMA-activation. However, GYY4137 can efficiently inhibit neutrophil activation by isolated IgG from ANCA patients.

### Sulfide's Effect of Neutrophil Degranulation

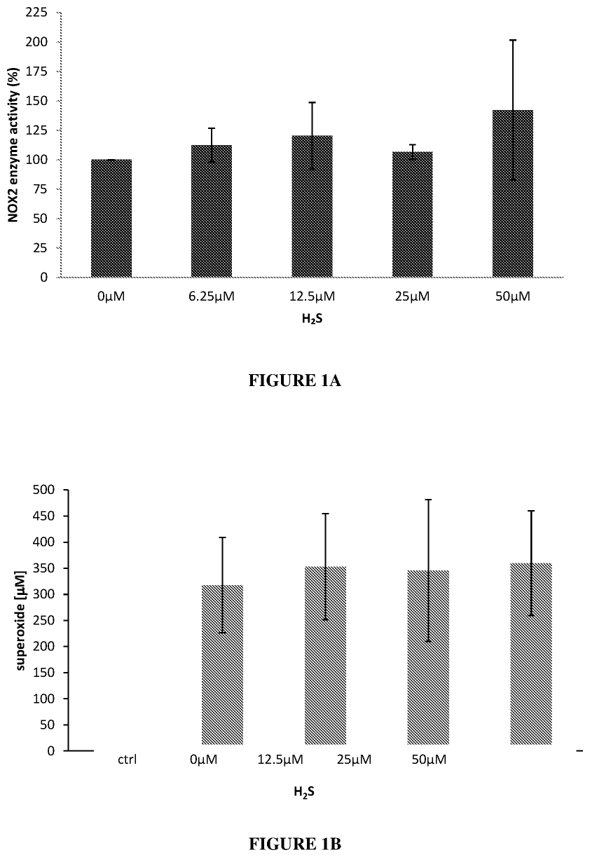

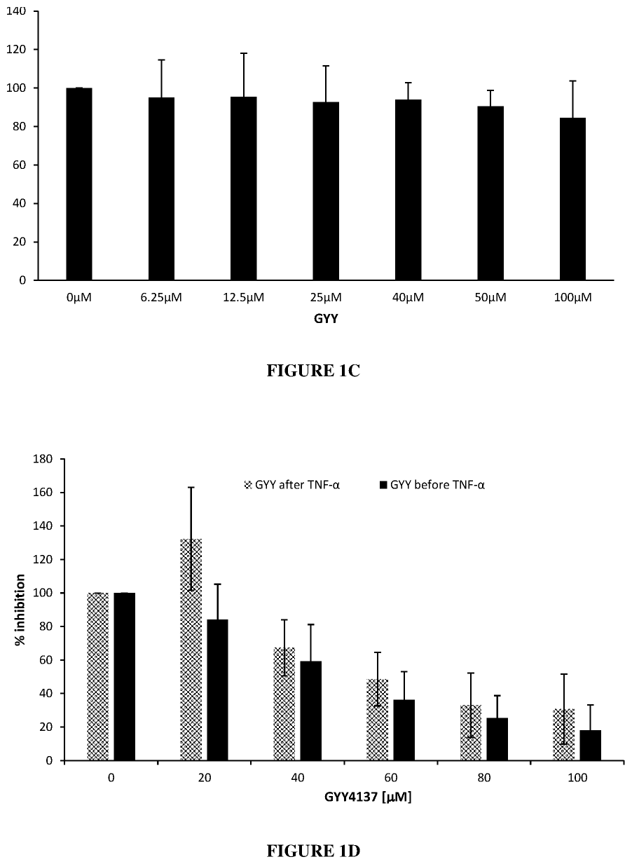

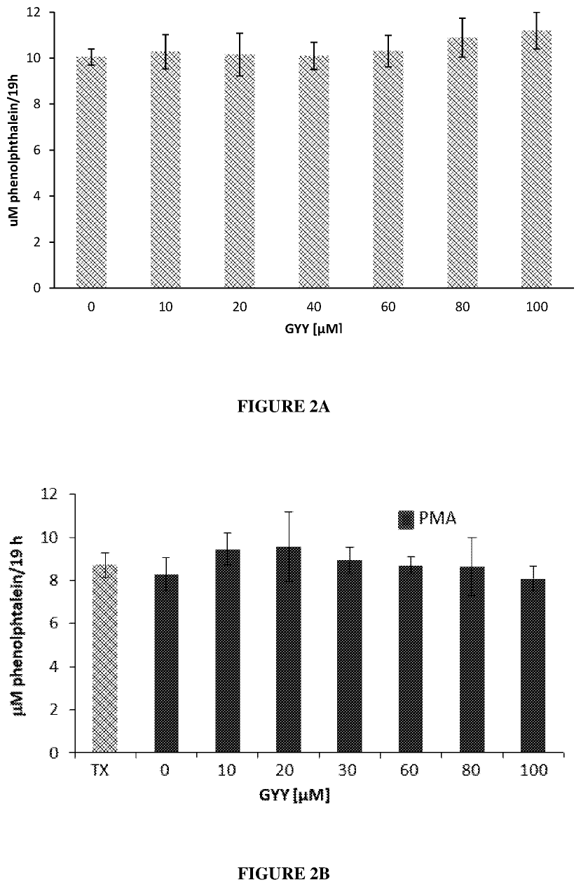

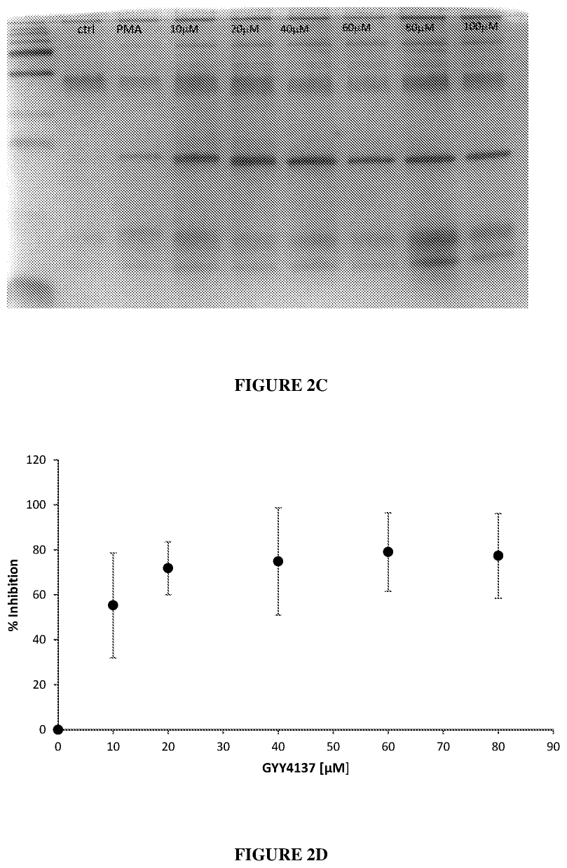

To investigate the effect of sulfide on the degranulation of neutrophils we used the β-glucuronidase assay, which is based on the liberation of phenolphthalein from phenolphthalein-glucuronide [Falk, R. J., et al., Anti—neutrophil cytoplasmic autoantibodies induce neutrophils to degranulate and produce oxygen radicals in vitro. Proc Natl Acad Sci USA, 1990. 87(11): p. 4115-9]. The interference of sulfide with the assay conditions was measured on neutrophil samples that were treated with 1% Triton X. Triton X was used to disrupt cell membranes and get maximal β-glucuronidase activity in supernatants. Results show that the average unit of enzyme activity that was calculated based on the amount of phenolphthalein (gg) liberated in 19 hours was 10.88 (±1.88) units. Data also indicates, that the addition of different concentrations of GYY4137 did not influence the enzyme activity or interfered with assay conditions (FIG. 2/A). PMA treated samples had similar β-glucuronidase activity in the supernatants and the presence of sulfide had again no effect on the enzymatic activity (FIG. 2/B). We further confirmed with SDS-PAGE followed by silver staining that the protein levels in the supernatants correlated well with the measured enzyme activities. Decrease in protein concentrations were not detected in GYY4137 treated samples again suggesting no interference of sulfide or GYY4137 with the assay (FIG. 2/C). In case of ANCA activation the degree of degranulation was similar to the Triton X or PMA treated samples with an average enzyme activity of 9.77 (±1.36) units. However, unexpectedly, in this case the presence of GYY4137 efficiently inhibited the liberation of phenolphthalein in a dose dependent manner with an IC₅₀ value of 6.47 μM (FIG. 2/D). Thus, surprisingly, the sulfide donor GYY4137 can efficiently inhibit neutrophil degranulation upon ANCA activation but had no effect on PMA-activation.

Effect of Sulfide on Neutrophil Priming with TNF-α

Inflammatory cytokines, like TNF-α, induce the translocation of ANCA antigens, such as MPO and PR3, to the cell surface, which primes the neutrophil cells for antibody-recognition-based activation. The present inventors have investigated the interference of sulfide on TNF-α-induced neutrophil priming by differential immunofluorescent labeling of the surface MPO using green (FIG. 3-2nd column) and the granular MPO using red (FIG. 3, 3rd column) fluorescent tags. In the black and white Figures fluorescence is shown in grey but can be identified via the position of the figure given herein. Samples that were treated with TNF-α translocated MPO to the membrane (FIG. 3, 2nd row) and no surface MPO could be detected in untreated control samples (FIG. 3, 1st row). Neutrophils that were treated with 25 μM or 50 μM GYY4137 5 minutes prior or together with the addition of TNF-α indicated the presence of diminished MPO signals on the membrane and more in azurophil granules (FIG. 3, 3rd-5th row). These results suggest that GYY4137 can inhibit TNF-α-induced translocation of ANCA antigen MPO and thereby neutrophil priming.

### Effect of Sulfide on Phagocytosis

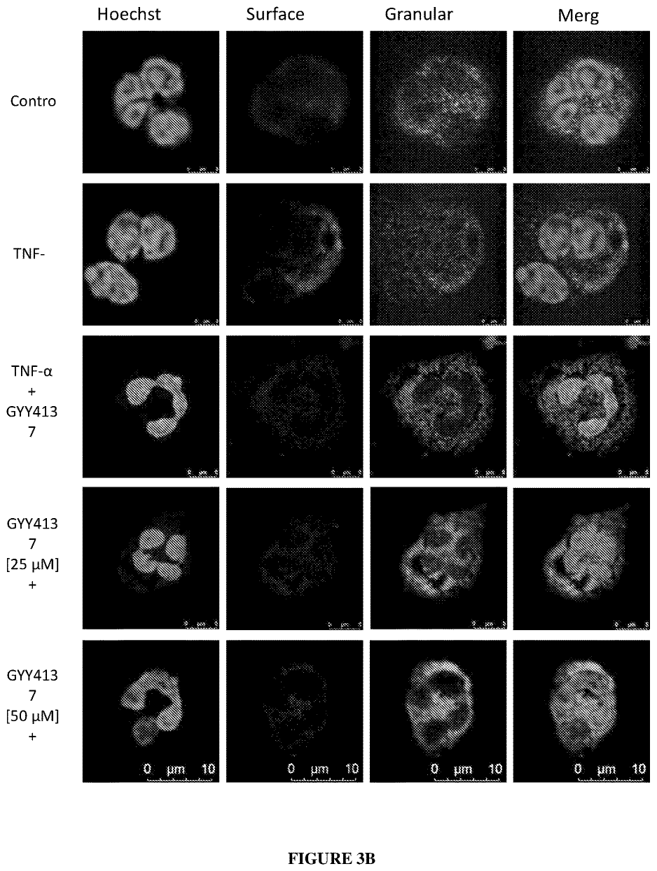

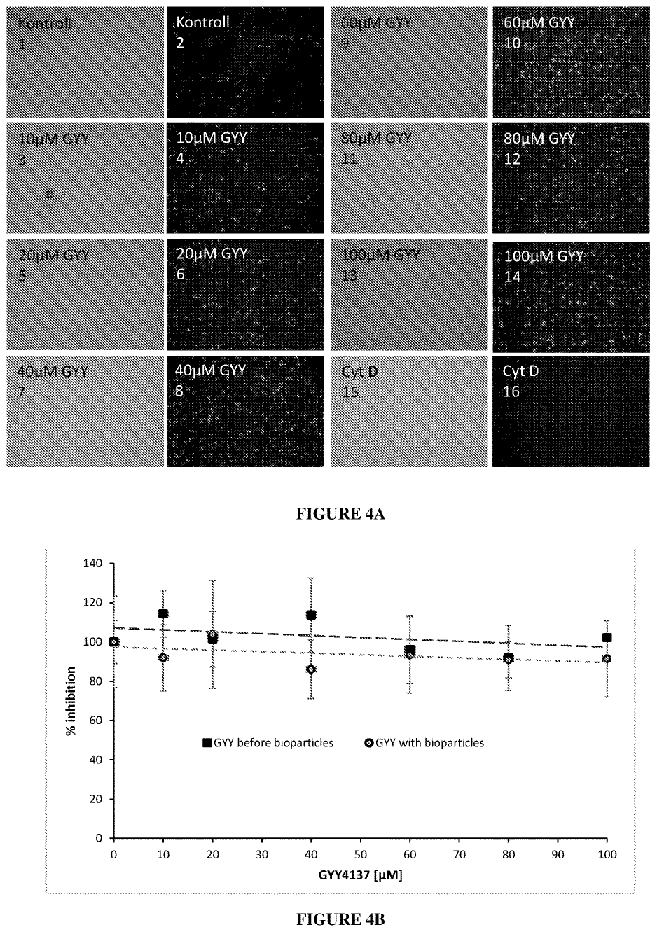

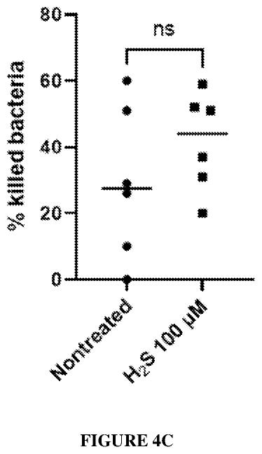

Control neutrophils have engulfed the E. coli Bioparticles (FIG. 4/A1-2) and this process was inhibited by cytochalasin D that inhibits phagocytosis by interfering with the formation of new actin filaments [Parod, R. J. and J. D. Brain, Immune opsonin-independent phagocytosis by pulmonary macrophages. J Immunol, 1986. 136(6): p. 2041-7](FIG. 4/A15-16). The addition of GYY4137 5 minutes prior or together with the bioparticles had no effect on phagocytosis (FIG. 4/A3-14). A plate assay was also used to quantitatively investigate the effects of sulfide on phagocytosis. Treatment with GYY4137 did not lower the reporting relative fluorescence signals at any given concentrations (20-100 μM) confirming the lack of inhibition of sulfide on phagocytosis (FIG. 4/B, black squares). Inhibition was also absent when the sulfide donor was given at the same time with the bioparticles (FIG. 4/B, gray circles). Thus, neutrophil phagocytosis is not inhibited in the presence of sulfide. Hydrogen sulfide also did not affect the bacterial killing of neutrophil graulocytes (FIG. 4/C).

The Sulfide Donor GYY4137 Protects from Kidney Damage in a Mouse Model of MPO-ANCA Vasculitis

There are several studies describing different ANCA mouse models (Animal Models of ANCA Associated Vasculitis). The method we chose to set up in our laboratory involves the adoptive transfer of activated B-cells of mice that were immunized with MPO or for control with BSA and non-immunized mice. Although this method does not result in a strictly autoimmune reaction, it provides a robust exhibition of symptoms similar to what can be observed in patients with ANCA-associated vasculitis [Xiao, H., et al., Antineutrophil cytoplasmic autoantibodies specific for myeloperoxidase cause glomerulonephritis and vasculitis in mice. J Clin Invest, 2002. 110(7): p. 955-63.]. Using this model, we investigated the effect of the hydrogen sulfide donor molecule GYY4137 on the emergence and severity of symptoms associated with AAV.

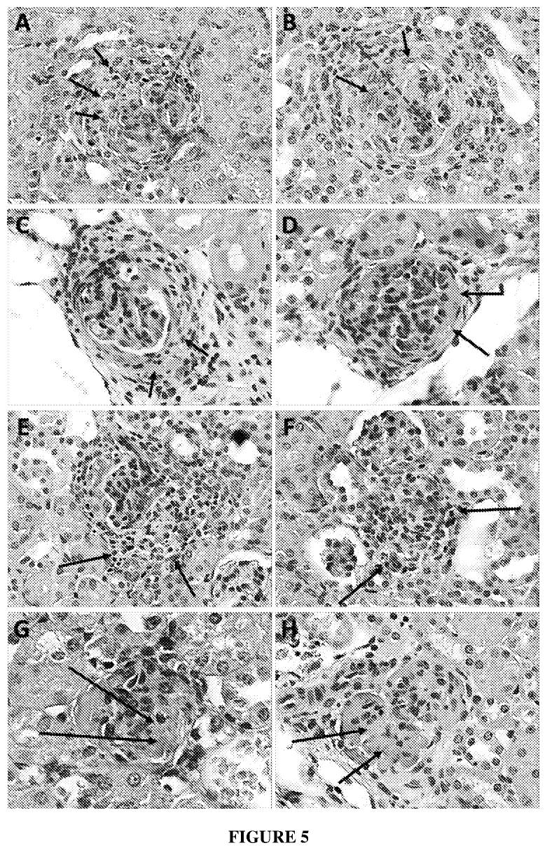

There is glomerular inflammation with necrotizing lesions where the GN is usually focal and segmental, but it may be also global and diffuse. Necrotizing lesions are invariably accompanied by crescents. Crescents are formed by proliferating parietal epithelial cells and monocytes; sometimes there are lymphocytes and granulocytes. The crescents, which contain only cells, without collagen, are called: cellular crescents (FIG. 5/A,B). The next step of the process is when fibroblasts, migrating to the Bowman's space, synthesize collagen that replaces progressively the crescents; when cellular components are mixed with collagen the lesions are called: fibrocellular crescents (FIG. 5/C); in advanced stages, without epithelial cells and only fibrous tissue, the lesion is called fibrous crescent (FIG. 5/D). These three stages indicate active or chronic lesions and therefore indicative of the response to treatment. Frequently there are extensive destructions of Bowman's capsules (FIG. 5/E.); in these cases it is usual to find granulomas (FIG. 5/F) and multinucleated cells surrounding the glomerulus, these granulomas do not indicate Wegener's granulomatosis. Glomerulosclerosis is scarring (sclerosis) of the glomeruli (FIG. 5/G,H). Glomerulosclerosis is caused by the activation of glomerular cells to produce scar material. This may be stimulated by molecules called growth factors, which may be made by glomerular cells themselves or may be brought to the glomerulus by the circulating blood that enters the glomerular filter.

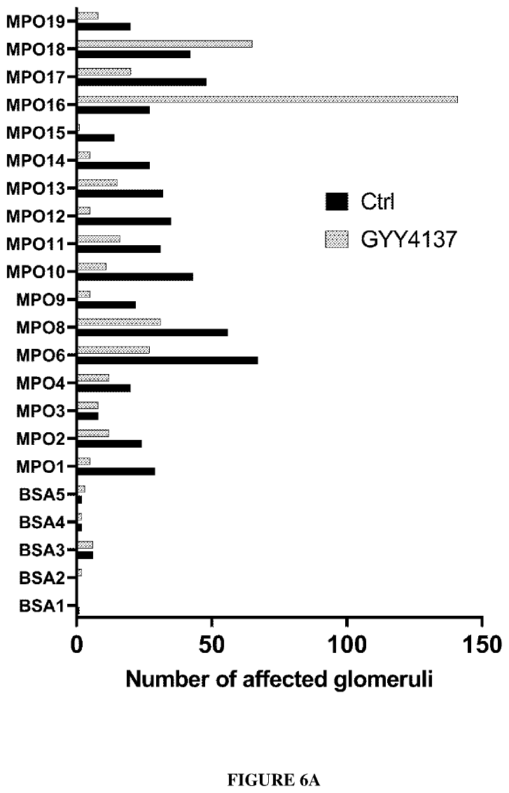

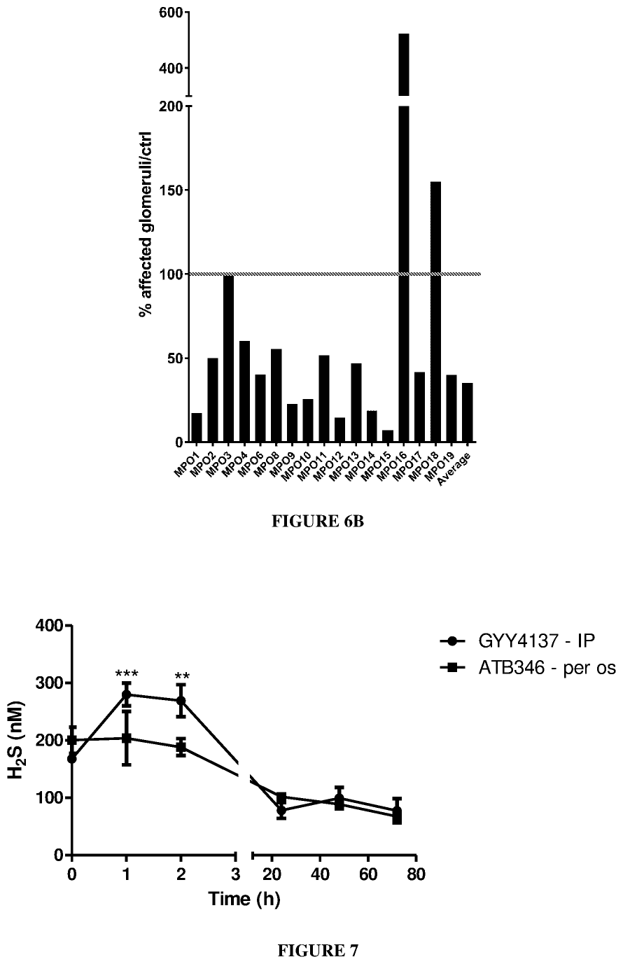

Rag 2 deficient mice that received splenocytes from control or BSA immunized MPO deficient mice did not develop inflammation in their kidneys. Rag 2 deficient mice that received splenocytes from MPO immunized MPO deficient mice developed symptoms that are described above with great variance (FIG. 6/A). The kidney samples of 2 different mouse pairs (MP016,MP018) showed that the GYY4137 treated samples had elevated levels of inflammation compared to the untreated samples. One individual from 2 different pairs (MPO5, MPO7) have passed away in 24 hours after intravenous injection of splenocytes, therefore we have excluded both pairs from the experiment. Semiquantitative comparative analysis of mouse pairs that developed symptoms upon adoptive transfer of splenocytes showed diminished symptoms of renal vasculitis as a result of GYY4137 treatment. Treated mice showed 64,9% lower number of affected glomeruli compared to their non treated pairs (excluding MPO3, MPO16 and MPO18) (FIG. 6/B).

### The H₂S Serum Level is Dependent on the Sulfide Donor Molecule, Dose and Administration

Normal (healthy) mice were treated with various sulfide donor preparations both intraperitoneally and orally:
GYY4137 was used in 150 and 750 μM/kg body weight,
A donor mixture (H₂S donor mix) was prepared which comprised GYY4137 (150 μmol/kg, 5 mg/ml strain), N-acetylcysteine (40 μmol/kg, 0.64 mg/ml strain) and pyridoxal 5′-phosphate (20 μmol/kg, 0.49 mg/ml strain).
ATB346 (otenaproxesul is present in Phase 2B efficacy trial; Antibe Therapeutics) was administered in 60 μmol/kg dose both orally and intraperitoneally. ATB346 (ATB in short) is a novel hydrogen sulfid-releasing derivative of naproxen with remarkably reduced toxicity, which also inhibits COX activity. ATB 346 is also an anti-inflammatory agent, at the same time a novel nonsteroidal anti-inflammatory drug (“NSAID”) that induces apoptosis of human melanoma cells.

ATB was tested in ANCA mouse model, administered orally (per os) and proved to be inefficient. In a further experiment ATB (60 μmol/kg) was administered to healthy mice orally to measure serum level in parallel with GYY4137 (150 μmol/kg) administered intraperitoneally and serum levels were compared. Surprisingly, while IP administered GYY4137, which proved to be active in ANCA-vasculitis mice model (see the example above as well as FIGS. 7 and 8), and increase H₂S serum level significantly, oral administered ATB346 has not increased H₂S serum level which indicates that elevated serum level correlates with the beneficial effect on the impaired kidney in AAV.

H₂S-Level was Measured from Sera of Animals

In a further experiment a sulfid donor mix (H₂S donor mix) was prepared which comprised GYY4137 (150 μmol/kg, 5 mg/ml strain), N-acetylcysteine (40 μmol/kg, 0.64 mg/ml strain) and pyridoxal 5′-phosphate (20 μmol/kg, 0.49 mg/ml strain). The components have been dissolved in PBS.

The H₂S donor mix was administered to healthy mice IP and per os. Serum concentration was measured at 0 h resulted in higher concentration of sulfide in the serum samples. Donor mix applied per os resulted in significant elevation of serum sulfide compared to IP treatment (FIG. 8/B). IP treatment did not increase the sulfide concentration in the serum of the animals despite containing 150 mol/kg GYY4137 (together with N-acetylcysteine and pyridoxal 5′-phosphate) (FIG. 8/A).

Thus, intraperitoneally administered GYY4137 has significantly increased H₂S serum level (see FIG. 7) whereas IP administered H₂S donor mix has not (FIG. 8A)—this finding was in close correlation with the fact that IP administered GYY4137 improved renal condition in ANCA vasculitis animal model whereas in case of IP administered donor mix no improvement was found.

Novel hydrogen sulfide donor GKK-895 was administered to healthy mice IP and serum concentrations were significantly elevated 2 h and 4 h after treatment compared to control and GYY4137 treated samples (FIG. 9). It was also demonstrated thatGKK-895, at much lower concentrations diminished renal symptoms of ANCA associated vasculitis to the same extent as higher concentrations of GYY4137 in the ANCA mouse model (FIG. 10).

These findings provide futrher evidence, that elevation of hydrogen sulfide concentration in the blood correlated with protective effects in the ANCA mouse model.

Taken together, the results lead to the conclusion that in fact any H₂S donor compounds, i.e. any compound which provides H₂S to a subject, i.e. elevates the serum level of H₂S, is plausibly useful in the treatment of MPO-ANCA vasculitis.

1. A H₂S donor compound for the treatment of antineutrophil cytoplasmic autoantibody (ANCA)-associated vasculitis (AAV) in a mammalian patient having AAV of the myeloperoxidase-ANCA (MPO-ANCA) serotype, preferably said AAV being a myeloperoxidase (MPO) autoantigen positive AAV wherein said subject has an increased MPO-specific ANCA level.

2. The H₂S donor compound according to claim 1 wherein said AAV is a myeloperoxidase (MPO) autoantigen positive AAV wherein said subject has an increased MPO-specific ANCA level.

3. The H₂S donor compound according to claim 1 wherein said H₂S donor compound is a slow H₂S releaser compound and said compound is used for preventing kidney damage and/or protecting the kidney in the mammalian subject having AAV,
inhibiting neutrophil granulocyte activation by anti-MPO antibodies of mammalian subject having AAV,
inhibiting neutrophil degranulation in the mammalian subject having AAV, and/or
inhibiting neutrophil priming in the mammalian subject having AAV,
in particular for preventing kidney damage and/or protecting the kidney in the mammalian subject having AAV.

4. The H₂S donor compound according to claim 1 wherein said AAV is selected from microscopic polyangiitis (MPA), granulomatosis with polyangiitis (GPA) (Wegener's granulomatosis), eosinophilic granulomatosis with polyangiitis (EGPA) (Churg-Strauss syndrome), and renal limited vasculitis.

4. (canceled)

5. The H₂S donor compound according to claim 1 wherein said compound is
administered orally to said mammalian patient, or
administered intraperitoneally to said mammalian patient, or.
administered intravenously to said mammalian patient,
preferably administered orally to said mammalian patient.

6. The H₂S donor compound according to claim 1 wherein said compound has general formula (Y)
M-L-Q, (Y)
wherein
Q is a H₂S releasing moiety which, in the body of a mammalian patient, releases H₂S into the blood stream of said mammalian patient,
wherein preferably
Q comprises a dithiole group (—S—S—), or
Q comprises a phosphinodithionate group (═P(S)S—)
L is an organic linking moiety,
M is a moiety which is covalently bound the rest of the molecule by a hydrolysable bond, wherein upon hydrolysis, M is converted to a compound which is tolerable by the mammalian patient, preferably beneficial to the mammalian patient,
wherein either L or M or none of them may be missing.

7. The H₂S donor compound according to claim 1 wherein said compound has general formula (X) 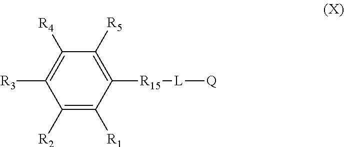
wherein
Q is a H₂S releasing moiety which, once administered to the mammalian patient, releases H₂S into the blood stream of said mammalian patient,
wherein preferably
Q comprises a 5 to 6 membered heterocycle comprising a dithiole group (—S—S—), or
Q comprises a phosphinodithionate group (═P(S)S⁻)
L is a linking moiety,
in a preferred embodiment L is C1-C8 alkylene, (preferably methylene), —, —O—, S, —NH—, an aryl, a C1-C4 alkylaryl or a 5 to 6 membered heterocycle which is optionally linked with Q via a C1-C4 alkylene,
wherein at least two, preferably three or four of R₁, R₂, R₃, R₄ and R₅ is H,
and R₁, R₂, R₃, R₄ and R₅ are, independently, selected from
H, halogen, pseudohalogen, —CN, —OH, —SH, —NO₂, —NH₂, —NHCH₃, —COOH, CONH₂,
substituted or unsubstituted C1-C8 alkyl, C2-C8 alkenyl, C2-C8 alkynyl, C1-C8 alkoxy, C1-C8 alkylamide, C6-C10 aryl, C7-C12 alkylaryl (aralkyl), 5 to 10 membered heteroaryl, 6-12 membered alkyl-heteroaryl, C1-C5 amide a C1-C8 carbonyl, (preferably a C2-C8 alkylcarbonyl, C3-C8 alkenylcarbonyl, C3-C8 alkynylcarbonyl,) a C1-C8 carboxyl, (preferably a C2-C8 alkylcarboxyl, C3-C8 alkenylcarboxyl or C3-C8 alkynylcarboxyl), a C2-C8 carboxylate ester (preferably a C2-C8 alkylester, C3-C8 alkenylester or C3-C8 alkynylester), said substituent, said substituent, if any, being selected from halogenide, pseudohalogenide, —OH, —SH, —OMe, —NO₂, —NH₂, —NHMe,
—OCOR₁₆, —COOR₁₇, —OR₁₈, —CONHR₁₉, wherein R₁₆, R₁₇ R₁₈ and R₁₉ are selected from H and substituted or unsubstituted C1-C8 alkyl, C2-C8 alkenyl, C2-C8 alkynyl, C1-C8 alkoxy, C1-C8 alkylamide, C6-C10 aryl, C7-C12 alkylaryl (aralkyl), 5 to 10 membered heteroaryl, 6-12 membered alkyl-heteroaryl, C1-C5 amide a C1-C8 carbonyl, (preferably a C2-C8 alkylcarbonyl, C3-C8 alkenylcarbonyl, C3-C8 alkynylcarbonyl,) a C1-C8 carboxyl, (preferably a C2-C8 alkylcarboxyl, C3-C8 alkenylcarboxyl or C3-C8 alkynylcarboxyl), a C2-C8 carboxylate ester (preferably a C2-C8 alkylester, C3-C8 alkenylester or C3-C8 alkynylester), said substituent, said substituent, if any, being selected from halogenide, pseudohalogenide, —OH, —SH, —OMe, —NO₂, —NH₂, —NHMe,
preferably R₁ and R₃ being independently selected from —OCOR₁₆, —COOR₁₇, —OR₁₈, wherein R₁₆, R₁₇ and R₁₈ are selected from H and substituted or unsubstituted C1-C8 alkyl, (preferably methyl or ethlyl) C2-C8 alkenyl, C2-C8 alkynyl, a C1-C8 carbonyl, (preferably a C2-C8 alkylcarbonyl, C3-C8 alkenylcarbonyl, C3-C8 alkynylcarbonyl,) a C1-C8 carboxyl, (preferably a C2-C8 alkylcarboxyl, C3-C8 alkenylcarboxyl or C3-C8 alkynylcarboxyl), a C2-C8 carboxylate ester (preferably a C2-C8 alkylester, C3-C8 alkenylester or C3-C8 alkynylester), said substituent, said substituent, if any, being selected from halogenide, pseudohalogenide, —OH, —SH, —OMe, —NO₂, —NH₂, —NHMe,
more preferably
wherein R₂, R₃ and R₅ are H,
R₁₅ is selected from C1-C8 alkylene, C1-C8 alkylether, C1-C8 carboxylate, preferably —(CH₂)ₙ—(CO)O— wherein n is 0, 1, 2 or 3, preferably 0 or 1,
or a pharmaceutically acceptable salt and/or solvate and/or complex thereof

8. The H₂S donor compound according to claim 1 wherein said compound has general formula I 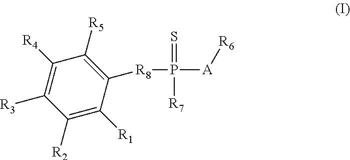
wherein
R₇ is selected from
a 3 to 10 membered, preferably 5 to 10 membered, more preferably a 5 to 6 membered heterocycle,
—NH—R₂₃ wherein R₂₃ is selected from C1-4 alkyl, C1-4 alkycarbonyl,
R₈ (preferably wherein R₅ is L) is selected from
—NH—
—CH₂—, —O—, S or NH, preferably CH₂, O or NH,
or the meaning of R₁₅ is nothing,
A is selected from
—S⁻, wherein if A is —S⁻, then R₆ is nothing,
—S— and —S⁻, wherein if A is —S— then
R₆ may be selected from H, a 5 to 10 membered, preferably a 6 membered, optionally substituted heterocycle, C1-4 alkyl or C6-C10 aryl, or
A is —S—, and R₆ and R₇ together with the other parts of the formula form a compound having the formula I.1 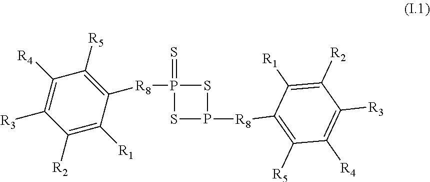
wherein R₁ to R₅ and R₈ are, independently, as defined above, wherein substituents of the two rings are either the same or different,
—O⁻
—O—, wherein if A is —O— then
R₆ may be selected from H, a 5 to 10 membered, preferably a 6 membered heterocycle, C1-4 alkyl, or C6-C10 aryl, or
a 10 to 20 membered organic moiety having one or two 5 to 6 membered heterocycles and optionally at least one 1 to 8 membered open chain moiety, optionally comprising 1 to 4, preferably 1 to 3 heteroatoms, and/or
R₆ is an organic moiety which, once hydrolysed, converted to a compound which is tolerable by the mammalian patient, preferably beneficial to the mammalian patient,
R₁, R₂, R₃, R₄ and R₅ are as defined above, or preferably R₁, R₂, R₃, R₄ and R₅ are, independently, selected from H, halogen, —CN, —OH, —SH, —NO₂, —NH₂, —NHCH₃, —COOH, CONH₂, wherein at least two of R₁, R₂, R₃, R₄ and R₅ is H;
preferably R₃ is —OCH₃ or each of R₁, R₂, R₃, R₄ and R₅ is H,
or a pharmaceutically acceptable salt and/or solvate and/or complex thereof,
in a particular embodiment a dichloromethane complex and/or a morpholinium salt.

9. The H₂S donor compound according to claim 1 wherein said compound has general formula I.2 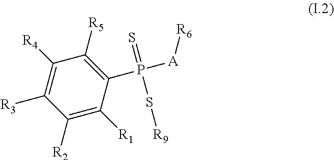
wherein
R₉ is H, wherein said H may be dissociated the —S—R₉ is —S⁻,
A is selected from nothing, and —O— and
wherein A is —O— then R₆ is
a 10 to 20 membered organic moiety having one or two 5 to 6 membered heterocycles and optionally at least one 1 to 8 membered open chain moiety, optionally comprising 1 to 4, preferably 1 to 3 heteroatoms, or wherein A is nothing then R₆ is
a 5 to 10 membered, preferably a 6 membered heterocycle, preferably an O and/or N-containing heterocycle, in particular a morpholino group connected via N,
R₁, R₂, R₃, R₄ and R₅, is selected from H, C1-4 alkyl, C1-4 alkoxy, halogenide, wherein at least two of R₁, R₂, R₃, R₄ and R₅ is H; preferably R₃ is —OMe, or each of R₁, R₂, R₃, R₄ and R₅ is H,
or a pharmaceutically acceptable salt and/or solvate and/or complex thereof;

10. The H₂S donor compound according to claim 9 wherein said compound has general formula I.3 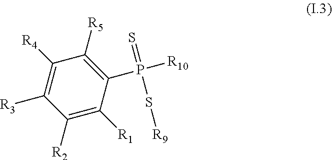
wherein R₁₀ is a 5 to 10 membered, preferably a 6 membered heterocycle, preferably an O and/or N-containing heterocycle, in particular a morpholino group connected via N,
R₁, R₂, R₃, R₃ and R₅ as well as R₉ are as defined above.

11. The H₂S donor compound fer-use according to claim 10, wherein preferably said compound has general formula I.3.1 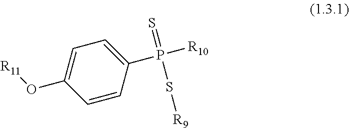
wherein R₁₁ is a C1-4 alkyl,
wherein preferably the compound for use according to claim 9 is GYY4137.

12. The H₂S donor compound according to claim 1, said compound having general formula II, 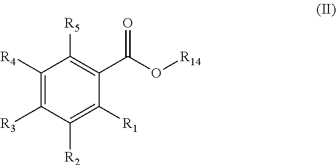
wherein R₁, R₂, R₃, R₄ and R₅ are as defined above
wherein at least two, preferably three or four of R₁, R₂, R₃, R₄ and R₅ is H,
wherein preferably R₁, R₂, R₃, R₄ and R₅ are, independently, selected from the group consisting of, (preferably R₁ is selected from the group consisting of
H, halogen, pseudohalogen, —CN, —OH, —SH, —NO₂, —NH₂, —NHCH₃, —COOH, CONH₂, preferably OH, and
—OCOR₁₆, —COOR₁₇, —OR₁₈, wherein R₁₆, R₁₇ R₁₈ and R₁₉ are selected from H and substituted or unsubstituted C1-C8 alkyl, C2-C8 alkenyl, C2-C8 alkynyl, C1-C8 alkoxy, C1-C8 alkylamide, C6-C10 aryl, C7-C12 alkylaryl (aralkyl), 5 to 10 membered heteroaryl, 6-12 membered alkyl-heteroaryl, C1-C5 amide a C1-C8 carbonyl, (preferably a C2-C8 alkylcarbonyl, C3-C8 alkenylcarbonyl, C3-C8 alkynylcarbonyl,) a C1-C8 carboxyl, (preferably a C2-C8 alkylcarboxyl, C3-C8 alkenylcarboxyl or C3-C8 alkynylcarboxyl), a C2-C8 carboxylate ester (preferably a C2-C8 alkylester, C3-C8 alkenylester or C3-C8 alkynylester), said substituent, said substituent, if any, being selected from halogenide, pseudohalogenide, —OH, —SH, —OMe, —NO₂, ONO₂, —NH₂, —NHMe,
preferably R₁ being selected from —OCOR₁₆, —COOR₁₇, —OR₁₈, wherein R₁₆, R₁₇ and R₁₈ are selected from H and substituted or unsubstituted C1-C8 alkyl, (preferably methyl or ethlyl) C2-C8 alkenyl, C2-C8 alkynyl, a C1-C8 carbonyl, (preferably a C2-C8 alkylcarbonyl, C3-C8 alkenylcarbonyl, C3-C8 alkynylcarbonyl,) a C1-C8 carboxyl, (preferably a C2-C8 alkylcarboxyl, C3-C8 alkenylcarboxyl or C3-C8 alkynylcarboxyl), a C2-C8 carboxylate ester (preferably a C2-C8 alkylester, C3-C8 alkenylester or C3-C8 alkynylester), said substituent, said substituent, if any, being selected from halogenide, pseudohalogenide, —OH, —SH, —OMe, —NO₂, —NH₂, —NHMe,
more preferably R₂, R₃, R₄ and R₅ are H, and R₁ is as define above, or
even more preferably R₁ is selected from —OH, —OCOR₁₆, wherein R₁₆ is selected from H and substituted or unsubstituted C1-C8 alkyl, (preferably methyl or ethyl) C2-C8 alkenyl, C2-C8 carboxylate ester (preferably a C2-C8 alkylester, C3-C8 alkenylester or C3-C8 alkynylester), said substituent, said substituent, if any, being selected from halogenide, pseudohalogenide, —OH, —SH, —OMe, —NO₂, ONO₂, —NH₂, —NHMe,
R₁₄ is a group having formula II1.2 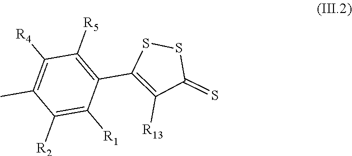
wherein in formula III.2
R₁, R₂, R₄ and R₅ are, independently, selected from H, halogen, pseudohalogen, —CN, —OH, —SH, —NO₂, —NH₂, —NHCH₃, —COOH, CONH₂, substituted or unsubstituted C1-C8 alkyl, C2-C8 alkenyl, C2-C8 alkynyl, C1-C8 alkoxy,
preferably H or OCH₃,
R₁₃ is selected from H and OCH₃, preferably

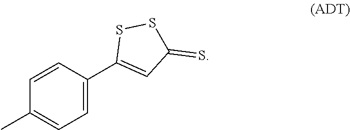

13. The H₂S donor compound according to claim 12, said compound being selected from

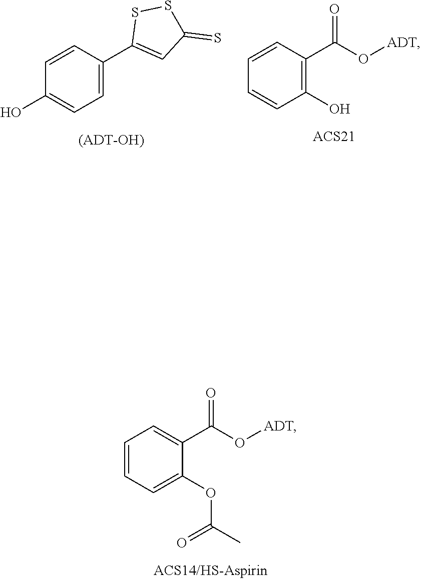

14. The H₂S donor compound according to claim 1, wherein said compound is a natural H₂S donor compound selected from allicin, alliin, diallil-trisulfide, diallil disulfide, diallil tetrasulfide, ajoene (e.g. E-ajoene or Z-ajoene), lenthionine, ovothiol, S-Allylmercaptocysteine (SAMC), 3H-1,2-Dithiole-3-thione, alpha lipoic acid.

15. The H₂S donor compound according to claim 1, wherein said compound comprises a substituted or unsubstituted, 5 membered heterocycle comprising a dithiole group (—S—S—, preferably an 1,2 dithiolane group or an 1,2 dithiolene group, preferably a group having the formula 5: 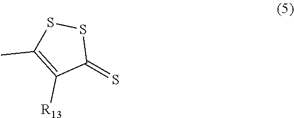
wherein R₁₃ is H or a C1-8 alkyl.

16. The H₂S donor compound for use according to claim 1 wherein said composition is administered daily, preferably one, two or three times a day, for at least one month, two months, three months, half year, one year or more.

17. A pharmaceutical composition comprising a H₂S donor compound according to claim 1, preferably according to any of claims 5 to 15, said composition being formulated for systemic administration.

18. The pharmaceutical composition comprising a H₂S donor compound according to claim 17,
said composition being formulated for oral administration, preferably in the form of capsules and/or tablets;
wherein preferably said composition is protected from light, moisture and decay to form H₂S during storage;
preferably said composition is formulated in a light-protective package.

19. The pharmaceutical composition comprising a H₂S donor compound according to claim 17, said composition being formulated for
intravenous administration, or for intraperitoneal administration; and
said composition is formulated as injection, or infusion.

20. The pharmaceutical composition comprising a H₂S donor compound for use according to claim 1,
said composition being formulated for topical administration,
preferably in the form of ointment, gel or emulsion,
said topical administration form comprising excipients to protect the H₂S donor compound moisture and/or decay to form H₂S during storage and optionally from light;
preferably said composition is formulated in a light-protective package.

21. The H₂S donor compound according to claim 1 wherein said compound increases bioavailable H₂S level in the serum of a mammalian subject after administration of said compound to said subject, preferably said subject having increased H₂S serum level after a time point of 1 h to 24 h after administration of said compound, if compared with normal H₂S serum level (or the serum level of H₂S without (or before) administration of said compound),
wherein preferably said increased H₂S serum level is at least two times of the normal serum level.
